(19) United States
(12) Patent Application Publication
(10) Pub. No.: US 2022/0233437 A1
(43) Pub. Date: Jul. 28, 2022

(54) DEVICES, COMPOSITIONS AND METHODS FOR COLONIC MICROBIOME ENGRAFTMENT

(71) Applicants: Thomas Julius BORODY, Five Dock (AU); Antony MILIS, Five Dock (AU); Annabel Kate CLANCY, Ashfield (AU)

(72) Inventors: Thomas Julius BORODY, Five Dock (AU); Antony MILIS, Five Dock (AU); Annabel Kate CLANCY, Ashfield (AU)

(21) Appl. No.: 17/615,241

(22) PCT Filed: May 29, 2020
(86) PCT No.: PCT/AU2020/050549

§ 371 Date: Nov. 30, 2021

Related U.S. Application Data
(60) Provisional application No. 62/855,099, filed on May 31, 2019.
(60) Provisional application No. 62/865,881, filed on Jun. 24, 2019.
(60) Provisional application No. 62/886,767, filed on Aug. 14, 2019.
(60) Provisional application No. 62/926,522, filed on Oct. 27, 2019.

(51) Int. Cl. A61K 9/08; A61K 31/166; A61K 33/44; A61K 35/24; A61K 38/46; A61K 9/00; A61P 31/04; A61M 3/02

(52) U.S. Cl. CPC A61K 9/08; A61K 31/166; A61K 33/44; A61K 35/24; A61K 2035/115; A61K 9/0031; A61P 31/04; A61M 3/0258; A61K 38/46

(57) ABSTRACT

Provided herein are compositions, including products of manufacture such as formulations, and kits, and methods, for treatment, amelioration or prevention of a disease, infection or condition caused or exacerbated by a pathological or abnormal in situ microbiome space, or a gastrointestinal (GI), disease, infection or condition or a disease or condition caused by, initiated by or exacerbated by a pathological microbiome, e.g., by a pathological GI or colonic microbiome residing in a gut mucosa.

### FIELD

This invention generally relates to gastroenterology and gastrointestinal (GI) microbiome biology and engraftment. In alternative embodiments, provided are products of manufacture (including colonic and small bowel formulation and fecal microbiota transplantation (FMT) delivery equipment) and compositions such as formulations, and kits, and methods, for treatment, amelioration or prevention of an in situ microbiome space, or a gastrointestinal (GI) disease, infection or condition or a disease or condition caused by, initiated by or exacerbated by a pathological microbiome, e.g., a pathological GI including a colonic microbiome, or a pathological microbiome on a GI mucosal surface. Applications of products of manufacture and methods as provided herein to other mucosal surfaces such as sinuses can help treat diseases of these surfaces, for example, sinusitis.

### BACKGROUND

The human gut microbiome can be viewed as a structured microbial community that operates like a microbial organ within the human host. Although small, an important part of this microbiome is its ability to colonize and thrive within the thin mucous layer that covers the colon epithelium. These mucosal microbes interact with the human intestinal tissues and other mucosal tissues and modulate human health. Attempts at replacing this ‘organ’ to treat human illnesses has evolved as fecal transplantation.

Faecal (Fecal) Microbiota Transplantation (FMT) has a long history going back to the 4th century in China and consists of the introduction of healthy donor rectal microbiota into the gastrointestinal (GI) tract of a person with an abnormal microbiota and clinical illness. Current methodology introducing microbiota into the bowel uses trans-colonoscopic or enema infusions, or otherwise delivery of FMT material to the small bowel through a long naso jejunal (NJ) delivery tube or swallowed capsules. Use of equipment or devices for ‘colonic’ delivery of FMT material has been described, for example, see Meron et al, U.S. Pat. No. 10,244,980.

Use of the FMT to treat Clostridioides difficile infection (CDI) (Clostridia was changed to Clostridioides by the Clinical and Laboratory Standards Institute (CLSI) in 2016) infection has been very successful, and this requires 1 or 2 enemas of human homogenized flora to achieve a 90% or even higher cure rate. A high level of engraftment has been demonstrated in such patients.

However, numerous conditions such as irritable bowel syndrome (IBS), ulcerative colitis (UC) and constipation are rarely cured by one or two or even multiple FMT GI infusions, where the introduced FMT appears to have difficulty in achieving engraftment.

By far the most difficult to cure with FMT appears to be constipation. Worldwide researchers are trying various methods of achieving implantation of fecal bacteria using multiple implantations or antibiotic pre-treatment, use of so-called “superdonors”, or using various methods of modifying the composition of the implanting flora, but to no avail. The implantation is not being achieved anywhere across the world. No matter what is attempted even multiple enemas of human stool generally fail to reverse constipation in any more than 20-30% of patients, even in those whom weekly infusions have taken place for more than 6 months.

Similarly, when trying to treat IBS, chronic abdominal pain of unknown origin, autism, and ulcerative colitis (UC) even recurrent enemas of normal human stool in well-prepared bowel linings fail to cure the conditions. In a colitis study where 81 patient's had FMT carried out 5 out of 7 days for 8 weeks only 2/81 patients were cured of UC in the long-term. Cure can mean both clinical and histological disappearance or absence of UC in patients off all therapies for more than about 12 or more months.

Biofilms are communities of organisms residing in a flat matrix or gel lining wet areas of the body in which the micro-organisms closely collaborate as a strategy for survival and persistence. Studies have demonstrated the presence of two general types of mucus gel secretion, or gut biofilm. One type is firmly adherent to the gut mucosal surface, and one is relatively sloppy or more loosely adherent, quite thick, and can be removed by suction or by use of water jets. It is the firmly adherent matrix or mucus gel which acts as a relatively stable protective barrier which may house pathogenic organisms including bacteria, fungi or viruses, acting as infectious agents, which can also maintain the disease or diseases such as Irritable Bowel Syndrome (IBS) caused by these organisms and their secretions originating in this matrix.

### SUMMARY

In alternative embodiments, provided herein are compositions and methods of therapy directed at removing pathogenic organisms (pathogens) residing in a mucosal matrix or a biofilm, as well as gastrointestinal (GI) luminal contents. In alternative embodiments, the pathogenic organisms are removed to prepare the GI environment for enhanced FMT material implantation, and hence to achieve immediate cure of the clinical condition caused by the residing pathogens. Provided are various compositions which can be used to remove these pathogens from the lumen and biofilms of the GI tract.

In various embodiments, provided are aqueous liquid formulations used alone or combined with any or all substances as described herein, the aqueous liquid formulations comprising:

(a) a soap, wherein optionally the soap is between about 1% to 95%, 5% to 90%, 10% to 80%, 15% to 70%, 20% to 60%, or 30% to 50%, or more, by weight or volume of the total aqueous liquid formulation,

wherein optionally the soap comprises a castile soap or equivalent or an IVORY™ soap or equivalent,

and optionally the soap comprises, or further comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide,

and optionally the soap further comprises: sodium tallowate, sodium cocoate, sodium palm kernelate, sodium chloride, sodium silicate, magnesium sulfate or any combination or mixture thereof,

and optionally the soap further comprises: coconut acid, palm kernel acid, tallow acid, palmitic acid, tetrasodium EDTA or any combination or mixture thereof,

and optionally the soap is diluted in water, saline or a super-oxidized aqueous solution (optionally OXUM™, MICRODACYN™, DERMACYN™),

and optionally the water comprises or is: a sterile water, a distilled water, tap water, an ozonated water, a hydrogen water (wherein optionally the hydrogen water is made by infusing hydrogen gas into water under pressure, and optionally the hydrogen water has between about 5 mg to 10 mg hydrogen per liter of water), an activated or electrolyzed water (optionally comprising sodium hydroxide and/or hypochlorous acid),

and optionally the saline comprises a hydrogen saline (wherein optionally the hydrogen saline is made by infusing hydrogen gas into saline under pressure, and optionally the hydrogen saline has between about 5 mg to 10 mg hydrogen per liter of saline), or a superoxygenated saline, or an about 0.9%, or between about 0.5% to 2%, or between about 0.25% to 4%, saline solution,

and optionally the ‘soap and water’ solution is a film-dissolving substance as well as a stool-removing product

and optionally the soap comprises a mixture of between about ¼ to 2 ounces (oz), or between about ⅛ to 3 oz, of soap, dissolved or mixed in 1 to 3 quarts, or in about 2 quarts, of water or saline, optionally a distilled water, a sterile water, tap water, or other water or saline as described herein, and with or without adding any one or more of the several agents described below:

(b) at least one compound, fluid, or composition comprising, or selected from the group consisting of:

(i) at least one biofilm disrupting agent,

wherein optionally the at least one biofilm disrupting agent comprises at least one enzyme,

wherein optionally the at least one enzyme comprises: a proteinase, an amylase, a lyase, a deoxyribonuclease (DNase), optionally dornase alpha, or PULMOZYME™, an alginase, a lyase or a glycoside hydrolase (optionally dispersin B);

(ii) at least one antibiotic,

and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone, which optionally is administered orally before the FMT (e.g., hours or days before the FMT) or into the bowel during the preparation of the bowel wall to implant the FMT,

and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;

(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-1-glutathione (GSNfaO), s-nitroso-N-acetylpenicillamine (SNAP), chlorhexidine, iodine, povidone-iodine (PI), (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water or saline, hydrogenated water or saline, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, iodine or povidone-iodine, (or WOKADINE™, PYODINE™, BETADINE™), boric or boronic acid, disodium EDTA, a phytocannabinoid, optionally cannabidiol (CBD), an alkyl dimethylol alkanate (ADMA), or any mixture or combination thereof;

(iv) at least one polyol or wetting agent,

and optionally the at least one polyol comprises xylitol, sorbitol, mannitol, erythritol, isomalt, maltitol syrup, lactitol, a hydrogenated starch hydrosylate or mixtures or combinations thereof,

wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;

(v) at least one surfactant or biosurfactant,

and optionally the at least one biosurfactant comprises: a probiotic, optionally a Bacillus strain, and optionally the Bacillus strain is Bacillus licheniformis,

and optionally the surfactant comprises an anionic, cationic, zwitterionic, or nonionic surfactant, or, any combination thereof,

and optionally the anionic surfactant comprises a sulfate, sulfonate or a phosphate ester,

and optionally the cationic surfactant comprises a tertiary amine or a quaternary ammonium salt,

and optionally the zwitterionic surfactant comprises a phospholipid, and optionally the phospholipid comprises a phosphatidylserine, phosphatidyl-ethanolamine, phosphatidylcholine or a sphingomyelin,

and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,

and optionally the surfactant comprises: a fatty acid esters of a sucrose or a sorbitol; a Tween; an alkyl polyglucoside; an amine or a phosphine oxide; a sulfoxide; or, any combination thereof;

(vi) at least one anti-quorum sensing (QS) compound, and optionally the at least one QS compound comprises: S-adenosyl-homocysteine, sinefungin, a N-sulfonyl homoserine lactone, or a synthetic derivative thereof, or a mixture or combination thereof; or

(vii) at least one prebiotic,

and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;

(viii) a stain and/or a dye,

wherein optionally the stain or dye comprises Coomassie Brilliant Blue, triarylmethane dye, rhodamine or erythrosine B, and optionally the stain or dye is infused between about 5 minutes (min) to 2 hours (hr), or between about 30 to 60 min, before a floral transplant procedure (wherein optionally the floral transplant procedure comprises a Fecal Microbiota Transplantation (FMT) in the gut);

(ix) a stool softening agent or a laxative,

wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;

(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;

(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;

(xii) pure (or substantially pure) or distilled water (H2O) (optionally alkaline or alkalized water), optionally used alone as a biofilm dissolver exploiting its hypotonic nature to penetrate bacteria resulting in swelling and bursting of the bacteria or other pathogen, and optionally the pure, alkaline, alkalized or distilled water is used as an enema, and optionally the enema or a colonic washing is by infusing the pure, alkaline, alkalized or distilled water by use of a colonic machine or equivalent, or by use of a naso-gastric (NG) long tube, or equivalent, and optionally ozone or ozonated water is administered after administration of the pure, alkaline, alkalized or distilled water;

(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;

(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,

and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,

and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent, and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,

and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,

and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;

(xv) a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;

(xvi) an ozonated water, optionally used alone as a biofilm-removing or biofilm-disrupting liquid, and optionally is administered using methods described in (xiv) for super-oxidized solutions (SOS);

(xvii) an ozone gas (which may damage a biofilm and/or its resident organisms, and optionally is administered as an insulated gas, optionally administered via a colonoscope or equivalent or by using gas bags or equivalent,

and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.

(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;

(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol optionally formulated as Carbowax™ GoLYTELY™, GlycoLax™, Fortrans™, TriLyte™, Colyte™, Halflytely™, Macrogol™ MiraLAX™, Plenvu™ (a formulation of: polyethylene glycol 3350, sodium ascorbate, sodium sulfate, ascorbic acid, sodium chloride and potassium chloride as an oral solution) or MoviPrep™ optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,

and optionally the Vitamin C or L-ascorbic acid is administered orally (optionally wash out luminal fecal material and to dissolve a biofilm simultaneously,

and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv), and/or

(xx) the at least one compound or composition comprises any combination of (i) to (xix).

In alternative embodiments, provided are powders or lyophilate formulations comprising a dried and powdered formulation, or a lyophilate, of an aqueous liquid formulation as provided herein, wherein the powder or the lyophilate formulation is capable of being reconstituted as a liquid formulation in an aqueous solution.

In alternative embodiments, provided are products of manufacture for, or containing comprising an aqueous liquid formulation as provided herein, or a powder or a lyophilate formulation as provided herein, wherein optionally the product of manufacture is a container, and optionally the container is a sachet.

In alternative embodiments, provided are methods for:
washing or lavaging the gut to remove substantially all or all biofilm or matrix that is adherent to an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) or a gut mucosa or luminal mucosa, wherein optionally the gut mucosa or luminal mucosa is a colon mucosa,
preparing a patient, or the patient's colon, for a fecal microbiota transplantation (FMT) administration, for or infusion or insertion of a living microbe or spore, wherein optionally the living microbe is a bacterium, fungi, virus, an Archea organism (Archaebacteria), or bacteriophage, and optionally the bacterium is cultured or recombinant bacterium, or
treating, ameliorating (including decreasing the symptoms of, or decreasing the severity of, or inhibiting progression of) or preventing: a disease, infection or condition caused or exacerbated by a pathological or abnormal in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue), or a disease, infection or condition or a disease or condition caused by, initiated by or exacerbated by a pathological microbiome space (e.g., GI or colonic) microbiome, for by a pathologic microbial organism,

wherein optionally a pathological microbial organism comprises: a bacteria, virus, fungi, Archea organism (Archaebacteria) such as Methano-brevibacter, or bacteriophage residing or being housed by an infected or abnormal in situ microbiome, e.g., a gut biofilm,

and optionally the infection, disease or condition is caused by a Clostridioides bacterium, and optionally the Clostridioides bacterium is C. difficile,

comprising administering or infusing into the in situ microbiome space, e.g., the gastrointestinal tract, of an individual in need thereof:

(a) an aqueous formulation or composition as provided herein, or

(b) an aqueous formulation comprising a soap,

wherein optionally the soap comprises a castile soap or equivalent or an IVORY™ soap or equivalent,

and optionally the soap comprises, or further comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide.

and optionally the soap further comprises: sodium tallowate, sodium cocoate, sodium palm kernelate, sodium chloride, sodium silicate, magnesium sulfate or any combination or mixture thereof,

and optionally the soap further comprises: coconut acid, palm kernel acid, tallow acid, palmitic acid, tetrasodium EDTA or any combination or mixture thereof,

and optionally the soap is diluted in water, saline or a super-oxidized aqueous solution (optionally OXUM™, MICRODACYN™, DERMACYN™), and optionally the water comprises or is: a sterile water, distilled water, tap water, ozonated water, activated or electrolyzed water (optionally comprising sodium hydroxide and/or hypochlorous acid), and optionally the saline comprises a superoxygenated saline or an about 0.9%, or between about 0.5% to 2%, or between about 0.25% to 4%, saline solution,

and optionally the soap comprises a mixture of between about ¼ to 2 ounces (oz), or between about ⅛ to 3 oz, of soap, dissolved or mixed in 1 to 3 quarts, or in about 2 quarts, of water, optionally a distilled water or a sterile water,

(c) an aqueous formulation comprising at least one compound or composition comprising, or selected from the group consisting of:

(i) at least one biofilm disrupting agent comprising at least one enzyme,

wherein optionally the at least one enzyme comprises: a proteinase, a lipase, an amylase, a deoxyribonuclease (DNase), optionally dornase alpha, or PULMOZYME™, an alginase, a lyase or a glycoside hydrolase (optionally dispersin B);

(ii) at least one antibiotic,

and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,

and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;

(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l-glutathione (GSNfaO), s-nitroso-N-acetylpenicillamine (SNAP), chlorhexidine, iodine, povidone-iodine (PI) (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water, hydrogenated water, activated or electrolyzed water, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, boric or boronic acid, disodium EDTA, a phytocannabinoid, optionally cannabidiol (CBD), an alkyl dimethylol alkanate (ADMA), or any mixture or combination thereof;

(iv) at least one polyol or a wetting agent,

and optionally the at least one polyol comprises xylitol, sorbitol, mannitol, erythritol, isomalt, maltitol syrup, lactitol, a hydrogenated starch hydrosylate, or mixtures or combinations thereof,

wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;

(v) at least one surfactant or biosurfactant,

and optionally the at least one biosurfactant comprises: a probiotic, optionally a Bacillus strain, and optionally the Bacillus strain is Bacillus licheniformis,

and optionally the surfactant comprises an anionic, cationic, zwitterionic, or nonionic surfactant, or, any combination thereof,

and optionally the anionic surfactant comprises a sulfate, sulfonate or a phosphate ester,

and optionally the cationic surfactant comprises a tertiary amine or a quaternary ammonium salt,

and optionally the zwitterionic surfactant comprises a phospholipid, and optionally the phospholipid comprises a phosphatidylserine, phosphatidyl-ethanolamine, phosphatidylcholine or a sphingomyelin,

and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,

and optionally the surfactant comprises: a fatty acid esters of a sucrose or a sorbitol; a Tween; an alkyl polyglucoside; an amine or a phosphine oxide; a sulfoxide; or, any combination thereof;

(vi) at least one anti-quorum sensing (QS) compound,

and optionally the at least one QS compound comprises: S-adenosyl-homocysteine, sinefungin, a N-sulfonyl homoserine lactone, or a synthetic derivative thereof, or a mixture or combination thereof; r

(vii) at least one prebiotic,

and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;

(viii) a stain and/or a dye,

wherein optionally the stain or dye comprises Coomassie Brilliant Blue, triarylmethane dye, rhodamine or erythrosine B;

(ix) a stool softening agent or a laxative,

wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;

(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;

(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;

(xii) pure (or substantially pure) or distilled water (H2O) (optionally alkaline water), optionally used alone as a biofilm dissolver exploiting its hypotonic nature to penetrate bacteria resulting in swelling and bursting of the bacteria or other pathogen, and optionally the pure, alkaline or distilled water is used as an enema, and optionally the enema or a colonic washing is by infusing infused the pure, alkaline or distilled water by use of a colonic machine or equivalent, or by use of a naso-gastric (NG) long tube, or equivalent, and optionally ozone or ozonated water is administered after administration of the pure, alkaline or distilled water;

(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;

(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy, and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,

and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,

and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,

and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,

and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;

(xv) a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;

(xvi) an ozonated water, optionally used alone as a biofilm-removing or biofilm-disrupting liquid, and optionally is administered using methods described in (xiv) for super-oxidized solutions (SOS);

(xvii) an ozone gas (which may damage a biofilm and/or its resident organisms, and optionally is administered as an insulated gas, optionally administered via a colonoscope or equivalent or by using gas bags or equivalent,

and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.

(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;

(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol optionally formulated as Carbowax™ GoLYTELY™, GlycoLax™, Fortrans™, TriLyte™, Colyte™, Halflytely™, Macrogol™ MiraLAX™, Plenvu™ (a formulation of: polyethylene glycol 3350, sodium ascorbate, sodium sulfate, ascorbic acid, sodium chloride and potassium chloride as an oral solution) or MoviPrep™ optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,

and optionally the Vitamin C or L-ascorbic acid is administered orally (optionally wash out luminal fecal material and to dissolve a biofilm simultaneously,

and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv); and/or

(xx) the at least one compound or composition comprises any combination of (i) to (xix); or

(d) an aqueous formulation comprising a soap of (b) formulated with or in combination with, or administered with, at least one compound or composition of (c).

In alternative embodiments of methods as provided herein: the administering or infusing into an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises administering or infusing the aqueous formulation of (a) (an aqueous formulation or composition as provided herein) or the aqueous formulation or composition of (b) (an aqueous formulation comprising a soap), or a combination thereof, via an oral route or an anal route,

and optionally the aqueous formulation of (a) (an aqueous formulation or composition as provided herein) or the aqueous formulation or composition of (b) (an aqueous formulation comprising a soap), or a combination thereof, is administered by use of a nasojejunal tube, a rectal speculum or a catheter, or a colonic tube or endoscope or equivalent thereof.

In alternative embodiments of methods as provided herein: the administering or infusing into the in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises one, two, three, four, five or six or more applications, washes or lavages of the aqueous formulation or composition as provided herein,

and optionally a sufficient amount of the aqueous formulation or composition as provided herein, is administered to remove between about 70% to 100%, 80% to 99.9%, or 85% to 99%, or 90% to 98%, or about 90%, 91%, 92%, 93%, 94%, 95%, 96%, 97%, 98% or 99% or more of the biofilm adherent to the wall or lumen of a gut, wherein optionally the wall or lumen of a gut comprises the wall of a colon,

and optionally between about between about 200 ml to about 1 to 5 liters (L), or about 0.5, 1, 2, 3, 4 or 5 or more liters, of liquid or formulation are infused into the individual in need thereof per treatment infusion, or application, wash or lavage,

and optionally each treatment infusion, or application, wash or lavage lasts between about 5 to 10 minutes and 10 hours, for example, between about 1 to 2 hours or 30 to 90 minutes,

and optionally the time between each treatment infusion, or application, wash or lavage is about 5 to 10 minutes and 10 hours, for example, between about 1 to 2 hours or 30 to 90 minutes, and optionally the administering or infusing of a formulation and/or a water or a saline into the in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises multiple infusions of about 10 cc to about 30 cc every about 1 to 6 hours (hrs), or over an about 24 to 48 hours period.

In alternative embodiments of methods as provided herein, exemplary methods comprise first administering an aqueous solution comprising a soap (e.g., one, two, three or more administrations of an aqueous solution comprising a soap), followed by an aqueous solution as provided herein not comprising any soap, for example, comprising water and N-acetyl-cysteine (NAC), a Vitamin C or L-ascorbic acid, or a polyethylene glycol optionally formulated as Carbowax™, GoLYTELY™, GlycoLax™, Fortrans™, TriLyte™, Colyte™, Halflytely™ Macrogol™, MiraLAX™, Plenvu™ or MoviPrep™, optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g, or an anti-persister cell therapy compound, or iodine or povidone-iodine (PI). In alternative embodiments, the aqueous solutions are administered one, two, three four, five or six or more times.

In alternative embodiments of methods as provided herein, exemplary methods comprise an aqueous solution comprising a soap and anti-persister cell therapy compound for patients whose previous treatments have failed.

In alternative embodiments of methods as provided herein, exemplary methods for the treatment of constipation comprise administration of an aqueous solution comprising a soap and a polyethylene glycol optionally formulated as Carbowax™, GoLYTELY™, GlycoLax™ Fortrans™, TriLyte™, Colyte™, Halflytely™, Macrogol™, MiraLAX™, Plenvu™ or MoviPrep™. In alternative embodiments, the aqueous solutions are administered one, two, three four, five or six or more times.

In alternative embodiments of methods as provided herein: the individual in need thereof is a human patient.

In alternative embodiments of methods as provided herein: the administering or infusing into the in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises administering the aqueous formulation as provided herein, or a water or a saline (including for example, a water or saline solution or formulation as provided herein), under pressure or in a liquid or formulation pulsating form.

In alternative embodiments of methods as provided herein: the administering or infusing into the gastrointestinal tract of an individual in need thereof comprises moving the individual in need thereof or massaging the gut of the individual in need thereof during and/or after administering of the aqueous formulation as provided herein.

In alternative embodiments of methods as provided herein: the administering or infusing into the in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) tract of an individual in need thereof comprises alternation (e.g., alternating cycles of) administering of: (i) a water, optionally a distilled, sterile, ozonated, hydrogenated water, or tap water, or, sterile saline, or a hydrogenated saline. and (ii) an aqueous formulation as provided herein or any one or several of the at least one compound, fluid, or composition as provided herein, including for example: biofilm disrupting or anti-biofilm agent or reagent; N-acetyl-cysteine (NAC); a super-oxidized solution (SOS); an anti-persister cell therapy compound; iodine or povidone-iodine (PI); a charcoal, a carbon or equivalent; a stool softening agent or a laxative; a Vitamin C or L-ascorbic acid; a prebiotic; an anti-quorum sensing (QS) compound; a polyol or wetting agent; an antibiotic; a stain and/or a dye; ozonated, pure, distilled or alkalized water,

wherein optionally the administering or infusing into the in situ microbiome space, e.g., gastrointestinal tract, of an individual in need thereof comprises one, two, three or more cycles of formulation and water enemas or administrations.

In alternative embodiments, methods as provided herein further comprise administration of at least one additional active agent, composition and/or fluid, wherein the at least one additional active agent, composition and/or fluid can be administered before, during and/or after administration of an aqueous liquid formulation as provided herein.

For example, alternative embodiments, the at least one additional active agent, composition and/or fluid, is administered in or as a capsule or tablet, e.g., as a pre-treatment capsule or tablet, or as a pre-treatment fluid or liquid.

In alternative embodiments, the at least one additional active agent, composition and/or fluid comprises:

(i) at least one biofilm disrupting or anti-biofilm agent or reagent,

wherein optionally the at least one biofilm disrupting or anti-biofilm agent or reagent comprises at least one enzyme,

wherein optionally the at least one enzyme comprises: a proteinase, an amylase, a lyase, a deoxyribonuclease (DNase), optionally dornase alpha or PULMOZYME™, an alginase, a lyase or a glycoside hydrolase (optionally dispersin B, or DspB);

(ii) at least one antibiotic,

and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,

and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;

(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l-glutathione (GSNfaO), s-nitroso-N-acetylpenicillamine (SNAP), chlorhexidine, a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, metallic silver or silver ions, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated or alkalized water or saline, hydrogenated water or saline, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, iodine or povidone-iodine (PI), (or WOKADINE™ PYODINE™ BETADINE™), boronic or boric acid, disodium EDTA, a phytocannabinoid, optionally cannabidiol (CBD), an alkyl dimethylol alkanate (ADMA), or any mixture or combination thereof;

(iv) at least one polyol or wetting agent,

and optionally the at least one polyol comprises xylitol, sorbitol, mannitol, erythritol, isomalt, maltitol syrup, lactitol, a hydrogenated starch hydrosylate or mixtures or combinations thereof, wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;

(v) at least one surfactant or biosurfactant,

and optionally the at least one biosurfactant comprises: a probiotic, optionally a Bacillus strain, and optionally the Bacillus strain is Bacillus licheniformis,

and optionally the surfactant comprises an anionic, cationic, zwitterionic, or nonionic surfactant, or, any combination thereof,

and optionally the anionic surfactant comprises a sulfate, sulfonate or a phosphate ester,

and optionally the cationic surfactant comprises a tertiary amine or a quaternary ammonium salt,

and optionally the zwitterionic surfactant comprises a phospholipid, and optionally the phospholipid comprises a phosphatidylserine, phosphatidyl-ethanolamine, phosphatidylcholine or a sphingomyelin,

and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,

and optionally the surfactant comprises: a fatty acid esters of a sucrose or a sorbitol; a Tween; an alkyl polyglucoside; an amine or a phosphine oxide; a sulfoxide; or, any combination thereof;

(vi) at least one anti-quorum sensing (QS) compound,

and optionally the at least one QS compound comprises: S-adenosyl-homocysteine, sinefungin, a N-sulfonyl homoserine lactone, or a synthetic derivative thereof, or a mixture or combination thereof; or

(vii) at least one prebiotic,

and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;

(viii) a stain and/or a dye,

wherein optionally the stain or dye comprises Coomassie Brilliant Blue, triarylmethane dye, rhodamine or erythrosine B,

and optionally the stain or dye is infused between about 5 minutes (min) to 2 hours (hr), or between about 30 to 60 min, before a floral transplant procedure (wherein optionally the floral transplant procedure comprises a Fecal Microbiota Transplantation (FMT) in the gut);

(ix) a stool softening agent or a laxative,

wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;

(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;

(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml; (xii) pure (or substantially pure) or distilled water (H2O) (optionally alkaline or alkalized water), optionally used alone as a biofilm dissolver exploiting its hypotonic nature to penetrate bacteria resulting in swelling and bursting of the bacteria or other pathogen, and optionally the pure, alkaline, alkalized or distilled water is used as an enema, and optionally the enema or a colonic washing is by infusing infused the pure, alkaline, alkalized or distilled water by use of a colonic machine or equivalent, or by use of a naso-gastric (NG) long tube, or equivalent, and optionally ozone or ozonated water is administered after administration of the pure, alkalized or alkaline or distilled water;

(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline or distilled water;

(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,

and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,

and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,

and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,

and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,

and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;

(xv) a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;

(xvi) an ozonated water, optionally used alone as a biofilm-removing or biofilm-disrupting liquid, and optionally is administered using methods described in (xiv) for super-oxidized solutions (SOS);

(xvii) an ozone gas (which may damage a biofilm and/or its resident organisms, and optionally is administered as an insulated gas, optionally administered via a colonoscope or equivalent or by using gas bags or equivalent,

and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.

(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;

(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol optionally formulated as Carbowax™ GoLYTELY™, GlycoLax™, Fortrans™, TriLyte™, Colyte™, Halflytely™, Macrogol™ MiraLAX™, Plenvu™ (a formulation of: polyethylene glycol 3350, sodium ascorbate, sodium sulfate, ascorbic acid, sodium chloride and potassium chloride as an oral solution) or MoviPrep™ optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,

and optionally the Vitamin C or L-ascorbic acid is administered orally (optionally wash out luminal fecal material and to dissolve a biofilm simultaneously,

and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv); and/or

(xx) any combination of (i) to (xix).

In alternative embodiments, methods as provided herein comprises alternative administration of formulations as provided herein and a water or a saline. For example, in one embodiment, to begin a treatment, a water such as a sterile water or hydrogenated water, or any solution that is hypotonic to bacteria, is first administered. Alternatively, an aqueous solution comprising vitamin C or ascorbic acid or equivalents, or iodine or povidone-iodine (PI) (or WOKADINE™, PYODINE™, BETADINE™), or any biofilm dissolving agent, is infused. This is followed by administration of a formulation as provided herein (comprising a soap) or a soap-comprising formulation; followed by another wash with a water or a saline, for example, by a wash with a hydrogenated water or an activated water; followed by another administration of a formulation as provided herein (comprising a soap) or a soap-comprising formulation, which optionally can further comprise at least one additional active agent, composition and/or fluid, e.g., as listed above, for example, comprising one or more antibiotics, vitamin C or ascorbic acid or equivalents, ozone, boronic or boric acid and/or iodine, povidone-iodine (PI), or any biofilm dissolving agent. As noted above, the first wash or treatment can be preceded by a pretreatment, e.g., by administration of a biofilm dissolving agent, for example, by administration of a biofilm dissolving agent in a capsule, tablet or liquid form.

In alternative embodiments, provided are kits comprising an aqueous liquid formulation as provided herein.

In alternative embodiments, provided are uses of an aqueous liquid formulation as provided herein or a kit as provided herein, for:
washing or lavaging an in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) to remove substantially all or all biofilm that is adherent to the in situ microbiome space, e.g., a gut mucosa or a luminal mucosa, wherein optionally the gut mucosa or luminal mucosa is a colon mucosa,
preparing the individual in need thereof, e.g., a patient, or the patient's colon, for a microbial transplantation, e.g., a fecal microbiota transplantation (FMT), administration, for or infusion or insertion of a living microbe or spore, wherein optionally the living microbe is a bacterium, fungi, Archea organism (Archaebacteria), or bacteriophage, and optionally the microbe or bacterium is cultured or recombinant microbe or bacterium, or
treating, ameliorating (including decreasing the symptoms of, or decreasing the severity of, or inhibiting progression of) or preventing: a disease, infection or condition or a disease or condition caused by, initiated by or exacerbated by a pathogen in an in situ microbiome space, or a gastrointestinal (GI) or other, disease, infection or condition or a disease or condition caused by, initiated by or exacerbated by a pathological or abnormal microbiome, e.g., GI or colonic microbiome, or by a pathologic microbial organism,

wherein optionally a pathological microbial organism comprises: a bacteria, a bacteriophage, a fungi, an Archea or a virus residing or being housed by an in situ microbiome space, e.g., a gut biofilm,

and optionally the infection, disease or condition is caused by a Clostridioides bacterium, and optionally the Clostridioides bacterium is C. difficile.

In alternative embodiments, provided are aqueous liquid formulation as provided herein or a kit as provided herein, for use in:
washing or lavaging the gut to remove substantially all or all biofilm that is adherent to a gut mucosa or luminal mucosa, wherein optionally the gut mucosa or luminal mucosa is a colon mucosa,
preparing an individual in need thereof, e.g., a patient, or the patient's colon, for an in situ microbiome space, e.g., a fecal microbiota transplantation (FMT), administration, for or infusion or insertion of a living microbe or spore, wherein optionally the living microbe is a bacterium, fungi, Archea organism (Archaebacteria), or bacteriophage, and optionally the microbe or bacterium is cultured or recombinant microbe or bacterium, or
treating, ameliorating (including decreasing the symptoms of, or decreasing the severity of, or inhibiting progression of) or preventing: a disease infection or condition caused by, initiated by or exacerbated by a pathologic microbe in an in situ microbiome space, e.g., a gastrointestinal (GI), disease, or a disease infection or condition or a disease infection or condition caused by, initiated by or exacerbated by a pathological microbiome, e.g., a pathologic GI or colonic microbiome, or by a pathologic microbial organism,

wherein optionally the pathological microbial organism comprises: a bacteria, bacteriophage, fungi, Archea or virus residing or being housed by an in situ microbiome space mucosa, e.g., a gut biofilm,

and optionally the infection is caused by a Clostridioides bacterium, and optionally the Clostridioides bacterium comprises a C. difficile.

In alternative embodiments, provided are pairs of pants or equivalent apparel for use with a colonic washout machine, bed or chair, wherein the pair of pants or shorts comprises: an orifice or opening sufficient to allow passage of a tube, optionally an enema tube or colonoscopic tube; and a clip, clamp or adhesive (or a plurality of clips, clamps or adhesives) or equivalent thereof capable of securing the tube to the pants or shorts such that the tube does not slip out of or through the orifice or opening, or is loose within the orifice or opening, and optionally the pair of pants or shorts is sufficiently tight-fitting such that it can hold the tube in place against a pressure or pulling force on the tube that, but without the tube being secured to the pants or shorts with the fitting, tie, snap, clip, clamp or adhesive or equivalent thereof, the tube would move through the orifice or opening, wherein optionally the pair of pants or shorts is made of, or comprises, an elastic material, wherein optionally the elastic material comprises elastane, SPANDEX™ or LYCRA™.

In alternative embodiments, provided are colonic washout machines or devices, wherein optionally the colonic washout machine or device is manufactured or configured in the form of a bed or a chair, optionally a movable bed or chair, and the colonic washout machine or device comprises a hole or opening approximate to the position of the anus of an individual sitting or laying on the bed, wherein the hole or opening is sufficiently large to allow passage of: a narrow rectal tube or equivalent (optionally the size of an endoscope); an endoscope or equivalent; an enema tube or equivalent (optionally between about 5 to 15 mm in diameter); or, a rectal speculum or a catheter, or a colonic tube, wherein the colonic washout machine or device further comprises:

(a) an auxiliary container or containers capable of holding a liquid or formulation attached to the colonic washout machine or device (optionally removably attached) and operatively attached or connected to the rectal speculum or catheter designed for insertion into a colon, and optionally the auxiliary container or containers are operatively attached or connected to the rectal speculum or catheter by use of a second tube,

and optionally the auxiliary container or containers further comprise a value or valves disposed between the auxiliary container or containers and the rectal speculum or catheter, wherein the value or valves are capable of controlling the rate of flow of liquid or formulation from the auxiliary container or containers to the rectal speculum or catheter, or the value or valves are capable of turning on or off the flow of liquid or formulation from the auxiliary container or containers to the rectal speculum or catheter,

and optionally the colonic washout machine or device further comprises a pump operably connected to the rectal catheter or equivalent, the auxiliary container or containers, or the second tube to move or facilitate the flow of liquid or formulation contents of the auxiliary container or containers out to a liquid or formulation flowing through the rectal catheter or equivalent,

and optionally the colonic washout machine or device further comprises a heater or heating unit to heat the liquid or formulation in the auxiliary container or containers to about body temperature,

and optionally the auxiliary container or containers or the second tube comprise a filter or filters capable of separating or straining particulate matter from a liquid or formulation before it is infused into the colon;

(b) a master container or containers capable of holding liquids or formulations for infusion into a colon, wherein optionally the master container or containers are operatively connected to the auxiliary container or containers of (a), and the master container or containers are operatively attached or connected to the rectal speculum or catheter designed for insertion into a colon, and optionally the master container or containers are operatively attached or connected to the endoscope or the rectal speculum or catheter by use of a third tube,

and optionally the master container or containers further comprise a value or valves disposed between the master container or containers and the rectal speculum or catheter, wherein the value or valves are capable of controlling the rate of flow of liquid or formulation from the master container or containers to the rectal speculum or catheter, or the value or valves are capable of turning on or off the flow of liquid or formulation from the master container or containers to the rectal speculum or catheter,

and optionally the colonic washout machine or device further comprises a pump operably connected to the rectal catheter or equivalent, the master container or containers, or the third tube to move or facilitate the flow of liquid or formulation contents of the master container or containers out to a liquid or formulation flowing through the rectal catheter or equivalent,

and optionally the colonic washout machine or device further comprises a heater or heating unit to heat the liquid or formulation in the master container or containers to about body temperature,

and optionally the master container or containers or the third tube comprise a filter or filters capable of separating or straining particulate matter from a liquid or formulation before it is infused into the colon;

(c) a pump operably connected to the rectal catheter or equivalent, the master container or containers, the third tube, the auxiliary container or containers and/or the second tube, where the pump when operational (turned on) moves or facilitates the flow of liquid or formulation contents of the rectal catheter or equivalent, the master container or containers, the third tube, the auxiliary container or containers and/or the second tube, to the colon,

and optionally the pump is a low pressure pump and/or a pressure adjustable pump, and optionally the pump further comprises a pressure gauge and pressure readings from the pump pressure gauge are transmitted to or are displayed to a reading device or screen on the colonic washout machine or device or remotely to a computer or a portable device, and optionally the portable device is a hand-held device or a smart phone, and optionally the pump is controlled remotely by use of a computer or a portable device or a foot pedal,

and optionally the pump is capable of providing a pulsating movement of liquid or formulation through the rectal catheter or equivalent into the colon, and optionally the pulsating movement of the pump is controlled remotely by use of a computer or a portable device or a foot pedal,

(d) a first set of motor or motors operably connected to rollers or equivalent for massaging, shaking or vibrating an individual lying on or sitting in the colonic washout machine or device,

and optionally the first set of motor or motors are controlled remotely by use of a computer or a portable device or a foot pedal,

(f) a second set of motor or motors operably connected to the colonic washout machine or device and capable of moving the colonic washout machine or device in a tipping or side to side movement,

and optionally the second set of motor or motors are controlled remotely by use of a computer or a portable device or a foot pedal; or

(g) any combination of (a) to (f).

In alternative embodiments, provided are rectal infusions or rectal aspiration tubes comprising a first end for inserting into the colon of an individual and a second end comprising a fitting for connection to a container or source of liquid or formulation for infusion into the colon, wherein the first end of the rectal infusion or rectal aspiration tube comprises a plurality of exit holes, orifices or openings to allow passages of fluids,

wherein the plurality of exit holes, orifices or openings are between about, or average from between about, 2 mm to 20 cm.

The details of one or more exemplary embodiments of the invention are set forth in the accompanying description below. Other features, objects, and advantages of the invention will be apparent from the description, and from the claims.

All publications, patents, patent applications cited herein are hereby expressly incorporated by reference for all purposes.

Forms of the invention include the following:
1. An aqueous liquid formulation comprising:

(a) a soap, wherein optionally the soap is between about 1% to 95%, 5% to 90%, 10% to 80%, 15% to 70%, 20% to 60%, or 30% to 50%, by weight or volume of the total aqueous liquid formulation,

wherein optionally the soap comprises a castile soap or equivalent or an IVORY™ soap or equivalent,

and optionally the soap comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide,

and optionally the soap further comprises: sodium tallowate, sodium cocoate, sodium palm kernelate, sodium chloride, sodium silicate, magnesium sulfate or any combination or mixture thereof,

and optionally the soap further comprises: coconut acid, palm kernel acid, tallow acid, palmitic acid, tetrasodium EDTA or any combination or mixture thereof,

and optionally the soap is diluted in water, saline or a super-oxidized aqueous solution (optionally OXUM™, MICRODACYN™, DERMACYN™),

and optionally the water comprises distilled water, tap water, ozonated water, a hydrogen water (wherein optionally the hydrogen water is made by infusing hydrogen gas into water under pressure, and optionally the hydrogen water has between about 5 mg to 10 mg hydrogen per liter of water), an activated or electrolyzed water (optionally comprising sodium hydroxide and/or hypochlorous acid), and optionally the saline comprises a superoxygenated saline or an about 0.9%, or between about 0.5% to 2%, or between about 0.25% to 4%, saline solution,

and optionally the soap comprises a mixture of between about ¼ to 2 ounces (oz), or between about ⅛ to 3 oz, of soap, dissolved or mixed in 1 to 3 quarts, or in about 2 quarts, of water, optionally a distilled water, and

(b) at least one compound or composition comprising, or selected from the group consisting of:

(i) at least one biofilm disrupting agent comprising at least one enzyme,

wherein optionally the at least one enzyme comprises: a proteinase, a lipase, an amylase, a deoxyribonuclease (DNase), optionally dornase alpha, or PULMOZYME™, an alginase, a lyase or a glycoside hydrolase (optionally dispersin B);

(ii) at least one antibiotic,

and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,

and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;

(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l-glutathione (GSNfaO), s-nitroso-N-acetylpenicillamine (SNAP), chlorhexidine, iodine, povidone-iodine (PI) (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water, hydrogenated water, activated or electrolyzed water, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, boric or boronic acid, disodium EDTA, a phytocannabinoid, optionally cannabidiol (CBD), an alkyl dimethylol alkanate (ADMA), or any mixture or combination thereof;

(iv) at least one polyol or a wetting agent,

and optionally the at least one polyol comprises xylitol, sorbitol, mannitol, erythritol, isomalt, maltitol syrup, lactitol, a hydrogenated starch hydrosylate, or mixtures or combinations thereof,

wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;

(v) at least one surfactant or biosurfactant,

and optionally the at least one biosurfactant comprises: a probiotic, optionally a Bacillus strain, and optionally the Bacillus strain is Bacillus licheniformis,

and optionally the surfactant comprises an anionic, cationic, zwitterionic, or nonionic surfactant, or, any combination thereof,

and optionally the anionic surfactant comprises a sulfate, sulfonate or a phosphate ester,

and optionally the cationic surfactant comprises a tertiary amine or a quaternary ammonium salt,

and optionally the zwitterionic surfactant comprises a phospholipid, and optionally the phospholipid comprises a phosphatidylserine, phosphatidyl-ethanolamine, phosphatidylcholine or a sphingomyelin,

and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,

and optionally the surfactant comprises: a fatty acid esters of a sucrose or a sorbitol; a Tween; an alkyl polyglucoside; an amine or a phosphine oxide; a sulfoxide; or, any combination thereof;

(vi) at least one anti-quorum sensing (QS) compound,

and optionally the at least one QS compound comprises: S-adenosyl-homocysteine, sinefungin, a N-sulfonyl homoserine lactone, or a synthetic derivative thereof, or a mixture or combination thereof; r

(vii) at least one prebiotic,

and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;

(viii) a stain and/or a dye,

wherein optionally the stain or dye comprises Coomassie Brilliant Blue, triarylmethane dye, rhodamine or erythrosine B;

(ix) a stool softening agent or a laxative, wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;

(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;

(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;

(xii) pure (or substantially pure) or distilled water (H2O) (optionally alkaline water), optionally used alone as a biofilm dissolver exploiting its hypotonic nature to penetrate bacteria resulting in swelling and bursting of the bacteria or other pathogen, and optionally the pure, alkaline or distilled water is used as an enema, and optionally the enema or a colonic washing is by infusing infused the pure, alkaline or distilled water by use of a colonic machine or equivalent, or by use of a naso-gastric (NG) long tube, or equivalent, and optionally ozone or ozonated water is administered after administration of the pure, alkaline or distilled water;

(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;

(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,

and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,

and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,

and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,

and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,

and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;

(xv) a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;

(xvi) an ozonated water, optionally used alone as a biofilm-removing or biofilm-disrupting liquid, and optionally is administered using methods described in (xiv) for super-oxidized solutions (SOS);

(xvii) an ozone gas (which may damage a biofilm and/or its resident organisms, and optionally is administered as an insulated gas, optionally administered via a colonoscope or equivalent or by using gas bags or equivalent,

and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.

(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;

(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol optionally formulated as Carbowax™ GoLYTELY™, GlycoLax™, Fortrans™, TriLyte™, Colyte™, Halflytely™, Macrogol™ MiraLAX™, Plenvu™ (a formulation of: polyethylene glycol 3350, sodium ascorbate, sodium sulfate, ascorbic acid, sodium chloride and potassium chloride as an oral solution) or MoviPrep™ optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,

and optionally the Vitamin C or L-ascorbic acid is administered orally (optionally wash out luminal fecal material and to dissolve a biofilm simultaneously,

and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv); and/or

(xx) the at least one compound or composition comprises any combination of (i) to (xix).
2. A powder or a lyophilate formulation comprising a dried and powdered formulation, or a lyophilate, of an aqueous liquid formulation of form 1, wherein the powder or a lyophilate formulation is capable of being reconstituted as a liquid formulation in an aqueous solution.
3. A product of manufacture for, comprising or containing therein an aqueous liquid formulation of form 1, or a powder or a lyophilate formulation of form 2, wherein optionally the product of manufacture is a container, and optionally the container is a sachet.
4. A method for:
   washing or lavaging an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) to remove substantially all or all biofilm that is adherent to the microbiome space (e.g., a gut mucosa or a luminal mucosa), wherein optionally the gut mucosa or luminal mucosa is a colon mucosa,
   preparing an individual in need thereof, e.g., a patient, or a patient's colon, for an in situ microbiome transplantation, e.g., a fecal microbiota transplantation (FMT), administration, for or infusion or insertion of a living microbe or spore, wherein optionally the living microbe is a bacterium fungi, Archea organism (Archaebacteria), or bacteriophage, and optionally the microbe or bacterium is a cultured or recombinant microbe or bacterium, or
   treating, ameliorating (including decreasing the symptoms of, or decreasing the severity of, or inhibiting progression of) or preventing: a disease, infection or condition caused or exacerbated by an in situ microbiome, e.g., a gastrointestinal (GI), or a disease, infection or condition or a disease or condition caused by, initiated by or exacerbated by a pathological microbiome, e.g., by a pathological GI or colonic microbiome, or by a pathologic microbial organism, wherein optionally a pathological microbial organism comprises: a bacteria, bacteriophage, fungi, Archea or virus residing or being housed by a gut biofilm,

and optionally the infection is caused by a Clostridioides bacterium, and optionally the Clostridioides bacterium is C. difficile,

comprising administering or infusing the in situ microbiota transplantation into or onto a tissue or an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof:

(a) an aqueous formulation or composition as set forth in form 1, or a powder or a lyophilate formulation of form 2,

(b) an aqueous formulation comprising a soap,

wherein optionally the soap comprises a castile soap or equivalent or an IVORY™ soap or equivalent,

and optionally the soap comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide.

and optionally the soap further comprises: sodium tallowate, sodium cocoate, sodium palm kernelate, sodium chloride, sodium silicate, magnesium sulfate or any combination or mixture thereof,

and optionally the soap further comprises: coconut acid, palm kernel acid, tallow acid, palmitic acid, tetrasodium EDTA or any combination or mixture thereof,

and optionally the soap is diluted in water, saline or a super-oxidized aqueous solution (optionally OXUM™, MICRODACYN™, DERMACYN™), and optionally the water comprises distilled water, tap water, ozonated water, hydrogenated water, activated or electrolyzed water (optionally comprising sodium hydroxide and/or hypochlorous acid), and optionally the saline comprises a superoxygenated saline or an about 0.9%, or between about 0.5% to 2%, or between about 0.25% to 4%, saline solution,

and optionally the soap comprises a mixture of between about ¼ to 2 ounces (oz), or between about ⅛ to 3 oz, of soap, dissolved or mixed in 1 to 3 quarts, or in about 2 quarts, of water, optionally a distilled water,

(c) an aqueous formulation comprising at least one compound or composition comprising, or selected from the group consisting of:

(i) at least one biofilm disrupting agent comprising at least one enzyme,

wherein optionally the at least one enzyme comprises: a proteinase, a lipase, an amylase, a deoxyribonuclease (DNase), optionally dornase alpha, or PULMOZYME™, an alginase, a lyase or a glycoside hydrolase (optionally dispersin B);

(ii) at least one antibiotic,

and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,

and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;

(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l -glutathione (GSNfaO), s-nitroso-N-acetylpenicillamine (SNAP), chlorhexidine, iodine, povidone-iodine (PI) (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water, hydrogenated water, activated or electrolyzed water, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, boric or boronic acid, disodium EDTA, a phytocannabinoid, optionally cannabidiol (CBD), an alkyl dimethylol alkanate (ADMA), or any mixture or combination thereof;

(iv) at least one polyol or a wetting agent,

and optionally the at least one polyol comprises xylitol, sorbitol, mannitol, erythritol, isomalt, maltitol syrup, lactitol, a hydrogenated starch hydrosylate, or mixtures or combinations thereof,

wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;

(v) at least one surfactant or biosurfactant,

and optionally the at least one biosurfactant comprises: a probiotic, optionally a Bacillus strain, and optionally the Bacillus strain is Bacillus licheniformis,

and optionally the surfactant comprises an anionic, cationic, zwitterionic, or nonionic surfactant, or, any combination thereof,

and optionally the anionic surfactant comprises a sulfate, sulfonate or a phosphate ester,

and optionally the cationic surfactant comprises a tertiary amine or a quaternary ammonium salt,

and optionally the zwitterionic surfactant comprises a phospholipid, and optionally the phospholipid comprises a phosphatidylserine, phosphatidyl-ethanolamine, phosphatidylcholine or a sphingomyelin,

and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,

and optionally the surfactant comprises: a fatty acid esters of a sucrose or a sorbitol; a Tween; an alkyl polyglucoside; an amine or a phosphine oxide; a sulfoxide; or, any combination thereof;

(vi) at least one anti-quorum sensing (QS) compound,

and optionally the at least one QS compound comprises: S-adenosyl-homocysteine, sinefungin, a N-sulfonyl homoserine lactone, or a synthetic derivative thereof, or a mixture or combination thereof; r

(vii) at least one prebiotic,

and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;

(viii) a stain and/or a dye, wherein optionally the stain or dye comprises Coomassie Brilliant Blue, triarylmethane dye, rhodamine or erythrosine B;

(ix) a stool softening agent or a laxative,

wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;

(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;

(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;

(xii) pure (or substantially pure) or distilled water (H2O) (optionally alkaline water), optionally used alone as a biofilm dissolver exploiting its hypotonic nature to penetrate bacteria resulting in swelling and bursting of the bacteria or other pathogen, and optionally the pure, alkaline or distilled water is used as an enema, and optionally the enema or a colonic washing is by infusing infused the pure, alkaline or distilled water by use of a colonic machine or equivalent, or by use of a naso-gastric (NG) long tube, or equivalent, and optionally ozone or ozonated water is administered after administration of the pure, alkaline or distilled water;

(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;

(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,

and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,

and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,

and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,

and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,

and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;

(xv) a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;

(xvi) an ozonated water, optionally used alone as a biofilm-removing or biofilm-disrupting liquid, and optionally is administered using methods described in (xiv) for super-oxidized solutions (SOS);

(xvii) an ozone gas (which may damage a biofilm and/or its resident organisms, and optionally is administered as an insulated gas, optionally administered via a colonoscope or equivalent or by using gas bags or equivalent,

and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.

(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;

(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol optionally formulated as Carbowax™ GoLYTELY™, GlycoLax™, Fortrans™, TriLyte™, Colyte™, Halflytely™, Macrogol™ MiraLAX™, Plenvu™ (a formulation of: polyethylene glycol 3350, sodium ascorbate, sodium sulfate, ascorbic acid, sodium chloride and potassium chloride as an oral solution) or MoviPrep™ optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,

and optionally the Vitamin C or L-ascorbic acid is administered orally (optionally wash out luminal fecal material and to dissolve a biofilm simultaneously,

and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv); and/or

(xx) the at least one compound or composition comprises any combination of (i) to (xix); or

(d) an aqueous formulation comprising a soap of (b) formulated with or in combination with, or administered with, at least one compound or composition of (c).
5. The method of form 4, wherein the administering or infusing into the in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises administering or infusing the aqueous formulation of 4(a) or the aqueous formulation or composition of 4(b), 4(c) or 4(d), or a combination thereof, via an oral, nasal or vaginal route or an anal route,

and optionally the aqueous formulation or composition of form 4 is administered by use of a nasojejunal tube or a rectal speculum or catheter, a colonic tube or an endoscope.
6. The method of form 4 or 5, wherein the administering or infusing into the in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises one, two, three, four, five or six or more applications, washes or lavages of the aqueous formulation of form 4(a) or the aqueous formulation or composition of form 4,

and optionally a sufficient amount of the aqueous formulation or composition of form 4 is administered to remove between about 70% to 100%, 80% to 99.9%, or 85% to 99%, or 90% to 98%, or about 90%, 91%, 92%, 93%, 94%, 95%, 96%, 97%, 98% or 99% or more of the biofilm adherent to the wall or lumen of the in situ microbiome space, e.g., the gut, wherein optionally the wall or lumen of a gut comprises the wall of a colon,

and optionally between about between about 200 ml to about 1 to 5 liters (L), or about 0.5, 1, 2, 3, 4 or 5 or more liters, of liquid are infused into the individual in need thereof per treatment infusion, or application, wash or lavage,

and optionally each treatment infusion, or application, wash or lavage lasts between about 5 to 10 minutes and 10 hours, for example, between about 1 to 2 hours or 30 to 90 minutes,

and optionally the administering or infusing of a formulation or water into the in situ microbiome space, e.g., gastrointestinal tract, of the individual in need thereof comprises multiple infusions of about 10 cc to about 30 cc every about 1 to 6 hours (hrs), or over a 24 to 48 hours period.
7. The method of any one of forms 4 to 6, wherein the individual in need thereof is a human patient.
8. The method of any one of forms 4 to 7, wherein the administering or infusing into the in situ microbiome space, e.g., gastrointestinal tract, of the individual in need thereof comprises administering the aqueous formulation or composition of form 4 under pressure or in a liquid pulsating form.
9. The method of any one of forms 4 to 8, wherein the administering or infusing into the gastrointestinal tract of an individual in need thereof comprises moving the individual in need thereof or massaging the gut of the individual in need thereof during and/or after administering of the aqueous formulation or composition of form 4.
10. The method of any one of forms 4 to 9, wherein the administering or infusing into the in situ microbiome space, optionally infusing into a gastrointestinal tract, of the individual in need thereof comprises alternation administering of: (i) water, optionally distilled water or sterile water, and (ii) an aqueous formulation or composition of form 4,

and optionally the aqueous formulation comprises any one or several of: biofilm disrupting or anti-biofilm agent or reagent; N-acetyl-cysteine (NAC); a super-oxidized solution (SOS); an anti-persister cell therapy compound; iodine or povidone-iodine (PI); a charcoal, a carbon or equivalent; a stool softening agent or a laxative; a Vitamin C or L-ascorbic acid; a prebiotic; an anti-quorum sensing (QS) compound; a polyol or wetting agent; an antibiotic; a stain and/or a dye; ozonated, pure, distilled or alkalized water,

wherein optionally the administering or infusing into the in situ microbiome space, e.g., gastrointestinal tract, of an individual in need thereof comprises one, two, three or more cycles of formulation and water enemas or administrations.
11. A kit comprising an aqueous liquid formulation of form 1, or an aqueous formulation as used in any of forms 4 to 10, or an aqueous liquid formulation of any or the preceding forms.
12. Use of an aqueous liquid formulation of form 1, or an aqueous formulation as used in any of forms 4 to 10, or an aqueous liquid formulation of any or the preceding forms, or a kit of form 11, for:
   washing or lavaging an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) to remove substantially all or all biofilm that is adherent to a microbiome space mucosa, e.g., a gut mucosa or a luminal mucosa, wherein optionally the gut mucosa or luminal mucosa is a colon mucosa,
   preparing an individual in need thereof, e.g., a patient, or a patient's colon, for an in situ microbiome space, e.g., a fecal microbiota transplantation (FMT), administration, for or infusion or insertion of a living microbe or spore, wherein optionally the living microbe is a bacterium, fungi, virus, bacteriophage or Archea, and optionally the bacterium or microbe is cultured or is a recombinant microbe or bacterium, or
   treating, ameliorating (including decreasing the symptoms of, or decreasing the severity of, or inhibiting progression of) or preventing: a disease, infection or condition caused or exacerbated by a microbiome space disease, e.g., a gastrointestinal (GI) disease, infection or condition or a disease or condition caused by, initiated by or exacerbated by a pathological microbiome, e.g., pathological GI or colonic microbiome, for by a pathologic microbial organism,

wherein optionally a pathological microbial organism comprises: a bacteria, bacteriophage, fungi, Archea or virus residing or being housed by a microbiome, e.g., gut, biofilm,

and optionally the infection is caused by a Clostridioides bacterium, and optionally the Clostridioides bacterium is C. difficile.
13. An aqueous liquid formulation of form 1, or an aqueous formulation as used in any of forms 4 to 10, or an aqueous formulation any or the preceding forms, or a kit of form 11, for use in:
   washing or lavaging the gut to remove substantially all or all biofilm that is adherent to an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) or gut mucosa or luminal mucosa, wherein optionally the gut mucosa or luminal mucosa is a colon mucosa,
   preparing a patient, or the patient's colon, for an in situ microbiome space transplantation, e.g., a fecal microbiota transplantation (FMT) administration, for or infusion or insertion of a living microbe or spore, wherein optionally the living microbe is a bacterium, bacteriophage, fungi, virus, Archea and optionally the microbe or bacterium is cultured or is a recombinant microbe or bacterium, or
   treating, ameliorating (including decreasing the symptoms of, or decreasing the severity of, or inhibiting progression of) or preventing: a microbiome space infection, disease or condition, e.g., a gastrointestinal (GI) disease, infection or condition or a disease or condition caused by, initiated by or exacerbated by a pathological microbiome space, GI or colonic microbiome, for by a pathologic microbial organism (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue),

wherein optionally a pathological microbial organism comprises: a bacteria bacteriophage, fungi, Archea or virus residing or being housed by an in situ microbiome space, e.g., a gut, biofilm,

and optionally the infection is caused by a Clostridioides bacterium, and optionally the Clostridioides bacterium is C. difficile,
14. A pair of pants or shorts for use with a colonic washout machine, bed or chair, wherein the pair of pants comprises: an orifice or opening sufficient to allow passage of a tube, optionally an enema tube or colonoscopic tube; and a clip, clamp or adhesive (or a plurality of clips, clamps or adhesives) or equivalent thereof capable of securing the tube to the pants or shorts such that the tube does not slip out of or through the orifice or opening, and optionally the pair of pants or shorts is sufficiently tight-fitting such that the pants or shorts can hold the tube in place against a pressure or pulling force on the tube that, but without the tube being secured to the pants or shorts with the fitting, tie, snap, clip, clamp or adhesive or equivalent thereof, the tube would move through the orifice or opening, wherein optionally the pair of pants or shorts is made of, or comprises, an elastic material, wherein optionally the elastic material comprises elastane, SPANDEX™ or LYCRA™
15. A colonic washout machine or device, wherein optionally the colonic washout machine or device is manufactured or configured in the form of a bed or a chair, optionally a movable bed or chair, and the colonic washout machine or device comprises a hole or opening approximate to the position of the anus of an individual sitting or laying on the colonic washout machine or device, wherein the hole or opening is sufficiently large to allow passage of a tube or device, optionally a rectal speculum or catheter, an endoscope, a colonoscope or a colonic tube,

wherein the colonic washout machine or device optionally further comprises:

(a) an auxiliary container or containers, or liquid or formulation holding modules, capable of holding a liquid or formulation attached to the colonic washout machine or device (optionally removably attached) and operatively attached or directly or indirectly connected to an endoscope, colonoscope or rectal speculum or catheter designed for insertion into a colon, and optionally the auxiliary container or containers are operatively attached or connected to the rectal speculum or catheter by use of a second tube,

and optionally the auxiliary container or containers further comprise a value or valves disposed between the auxiliary container or containers and the rectal speculum or catheter, wherein the value or valves are capable of controlling the rate of flow of liquid or formulation from the auxiliary container or containers to the rectal speculum or catheter, or the value or valves are capable of turning on or off the flow of liquid or formulation from the auxiliary container or containers to the rectal speculum or catheter,

and optionally the colonic washout machine or device further comprises or incorporates one or more intake ports, cassette spaces or cavities, each which can take (or input) a cassette, cartridge or a removable cartridge housing, and optionally the removable cartridge housing is fitted to have inserted therein a cassette or a cartridge,

and optionally the cassette or cartridge comprises or has contained therein a liquid, formulation, powder or lyophilate formulation for dissolving a biofilm; a drug or an active agent; or, an FMT material,

and optionally the colonic washout machine or device further comprises a pump or pumps operably connected to the rectal speculum or catheter, the auxiliary container or containers, or the second tube to move or facilitate the flow of liquid or formulation contents of the auxiliary container or containers out to a liquid or formulation flowing through the rectal speculum or catheter,

and optionally the pump or pumps are capable or programmed to create a waterhammer effect, which is created by a rapid back and forth movement of a water or liquid or formulation column for a short distance, for example, for a distance of between about 0.5 to 10 cm, or between about 1 mm to 5 cm, and the pump or pumps comprise or use a piston-driven system or equivalent such that a water or liquid column is induced to have a back-and-forth movement in situ,

and optionally the colonic washout machine or device further comprises a warming module, heater or heating unit to heat a fluid or liquid or formulation in the auxiliary container or containers, or the tubes, to about body temperature, and optionally the device (optionally the one or more intake ports, or cavities, or cassette or cartridge housings) further comprise an thermometer or thermostat capable of reading the temperature of a liquid or formulation, and thermometer or thermostat are capable of controlling the temperature of the liquid or formulation, where the liquid or formulation or fluid can be warmed and/or maintained at approximate body temperature, or the liquid or formulation or fluid can be warmed and/or maintained at about 37° C., and optionally the thermometer or thermostat is remotely read and/or is remotely controlled, for example, by a hand-held device or a computer,

and optionally the auxiliary container or containers, the cassette or cartridge, the cassette or cartridge housing, or the second tube, comprise a filter or filters capable of separating or straining a particulate matter from a liquid or formulation before it is infused into the colon, and optionally the auxiliary container or containers, the cassette or cartridge, the cassette or cartridge housing, or the second tube, comprise fitting that allow removal or exchange of or insertion of a new filter or filters;

(b) a master container or containers capable of holding liquids or formulations for infusion into a colon, wherein optionally the master container or containers are operatively connected to the auxiliary container or containers of (a), and the master container or containers are operatively attached or connected to a rectal speculum or catheter, and optionally the master container or containers are operatively attached or connected to the rectal speculum or catheter by use of a third tube,

and optionally the master container or containers further comprise a value or valves disposed between the master container or containers and the rectal speculum or catheter, wherein the value or valves are capable of controlling the rate of flow of liquid or formulation from the master container or containers to the rectal speculum or catheter, or the value or valves are capable of turning on or off the flow of liquid or formulation from the master container or containers to the rectal speculum or catheter,

and optionally the colonic washout machine or device further comprises a pump operably connected to the rectal speculum or catheter, the master container or containers, or the third tube to move or facilitate the flow of liquid or formulation contents of the master container or containers out to a liquid or formulation flowing through the rectal speculum or catheter,

and optionally the colonic washout machine or device further comprises a heater or heating unit to heat the liquid or formulation in the master container or containers to about body temperature, or to about 37° C.,

and optionally the master container or containers or the third tube comprise a filter or filters capable of separating or straining particulate matter from a liquid or formulation before it is infused into the colon;

(c) a pump operably connected to the rectal speculum or catheter, the master container or containers, the third tube, the auxiliary container or containers and/or the second tube, where the pump when operational (turned on) moves or facilitates the flow of liquid or formulation contents of the rectal speculum or catheter, the master container or containers, the third tube, the auxiliary container or containers and/or the second tube, to the colon,

and optionally the pump is a low pressure pump and/or a pressure adjustable pump, and optionally the pump further comprises a pressure gauge and pressure readings from the pump pressure gauge are transmitted to or are displayed to a reading device or screen on the colonic washout machine or device or remotely to a computer or a portable device, and optionally the portable device is a hand-held device or a smart phone, and optionally the pump is controlled remotely by use of a computer or a portable device or a foot pedal,

and optionally the pump is capable of providing a pulsating movement of liquid or formulation through the rectal speculum or catheter into the colon, and optionally the pulsating movement of the pump is controlled remotely by use of a computer or a portable device or a foot pedal,

(d) a first set of motor or motors operably connected to rollers or equivalent for massaging, shaking or vibrating an individual lying on or sitting in the colonic washout machine or device,

and optionally the first set of motor or motors are controlled remotely by use of a computer or a portable device or a foot pedal,

(f) a second set of motor or motors operably connected to the colonic washout machine or device and capable of moving the colonic washout machine or device in a tipping or side to side movement,

and optionally the second set of motor or motors are controlled remotely by use of a computer or a portable device or a foot pedal; or

(g) any combination of (a) to (f).
16. A rectal infusion or rectal aspiration tube or speculum comprising a first or distal end for inserting into the colon of an individual and a second proximal end comprising a fitting for connection to a container or source of liquid or formulation for infusion into the colon, wherein the first end of the rectal infusion or rectal aspiration tube comprises a plurality of exit holes, orifices or openings to allow passages of fluids,

wherein the plurality of exit holes, orifices or openings are between about, or average from between about, 2 mm to 20 cm, and optionally the plurality of exit holes, orifices or openings are milled or made by a 3D printer,

and optionally the plurality of openings are positioned at about 1, 2, and 3 cm from the distal tip, or a plurality of openings are positioned between about 0.5 to 20, or 1 to 10 cm from the distal tip of the rectal infusion or rectal aspiration tube, or speculum.

### BRIEF DESCRIPTION OF THE DRAWING

The drawing set forth herein is illustrative of at least an exemplary embodiment provided herein and is not meant to limit the scope of the invention as encompassed by the claims.

The drawing is not done to scale, it is only for displaying an exemplary embodiment of the present invention.

FIG. 1 schematically illustrates an exemplary colonic machine as disclosed herein.

### DETAILED DESCRIPTION

In alternative embodiments, provided are compositions, including products of manufacture such as such as formulations, and kits, and methods, for treatment, amelioration (including decreasing the symptoms of, or decreasing the severity of, or inhibiting progression of) or prevention of disease, infection or condition caused or exacerbated by a microbe in an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) or a disease, infection or condition or a disease, infection or condition caused by, initiated by or exacerbated by a pathological microbiome, e.g., pathological GI or colonic microbiome, for example by a pathological microbial organism such as a bacteria, bacteriophage, fungi, Archea or virus residing or being housed by a microbiome mucosa, e.g., a gut biofilm.

Provided herein are methods and compositions for removing and/or breaking up the mucous layer, or biofilm, which can line an in situ microbiome space mucosa, e.g., a gut or lumen wall, which has been described as “matrix-enclosed” mixed populations of bacteria, bacteriophage, fungi, viruses and/or Archea that adhere to or reside in biotic and abiotic surfaces in or on a mucosa, e.g., in the gut. In alternative embodiments, methods and compositions as provided herein can (and are used to) completely or substantially breakup and/or dissolve the biofilm that covers an in situ microbiome space mucosa (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue), e.g., the GI (e.g., the colonic) mucosa; this permits the successful colonic implantation of donor flora or cultured consortium of bacteria (e.g., FMT), provided the incoming microflora is delivered to the biofilm-free host mucosa within about 10 to 20 minutes (min), which facilitates growth of the healthy biofilm in the implantation material (e.g., the FMT). Delay may permit any remaining cells from the old biofilm to regrow and maintain the previous illness. Hence, rapid insertion of the FMT material can prevent or substantially lower the chance that the original pathogen (washed off with the biofilm using compositions and/or methods as provided herein) will re-grow on the biofilm.

In alternative embodiments, provided are compositions, including products of manufacture such as such as formulations, and kits, and methods, for achieving an efficient microbiota transplantation engraftment in situ, including in a gut (optionally including stomach, intestine and/or colon), sinus, vaginal, oral mucosa, tongue, stomach, skin, bladder, urethral, ureter, ear, bronchial, trachea, pharynx, lung, sinuses, lung or other microbiome space or microbiome-comprising tissue. The inventor has found that previous processes of simply infusing into the colon (in the gastrointestinal (GI) engraftment) a fecal flora (e.g., a Fecal Microbiota Transplantation (FMT)) for implantation or transplantation fails to take into consideration the firmly adherent matrix or mucus which acts as a layer which blocks engraftment of the floral implant or transplant (similarly, simply infusing into the sinus, vaginal, oral mucosa, tongue, stomach, skin, bladder, urethral, ureter, ear, bronchial, trachea, pharynx, lung, sinuses, lung or other microbiome space or microbiome-comprising tissue encounters the same problem—a firmly adherent host-derived matrix or mucus acts as a layer which blocks engraftment of the floral implant or transplant into that microbiome space). Hence, one needs to firstly remove this obstructing layer to permit access of the implanted microbiome to the human tissues, e.g., gut (optionally including stomach, intestine and/or colon), sinus, vaginal, bronchial, lung or other microbiome space), vagina, oral mucosa, tongue, stomach, skin, bladder, urethral, ureter, ear, bronchial, trachea, pharynx, lung, sinuses, bronchia, lung or other microbiome space, or any tissue or mucosal cells comprising a microbiome, which can then rapidly generate a new and healthy biofilm. However, achieving removal of the firmly adherent matrix is the challenge previously unsolved, possibly because the process is complex and demands that mechanical and chemical methods be combined to prepare the gut mucosal cells to receive the FMT material. Provided for the first time herein is are compositions and processes (in alternative embodiments, a three-step process) that completely or substantially remove the obstructing adherent matrix or mucosal layer to permit access of the implanted microbiome to the human tissues (the targeted microbiome space) for rapid generation of a new and healthy biofilm. In alternative embodiments, the three-step process comprises first cleansing the colon of all or substantially most stool; followed by removing all or substantially all of the firmly adherent matrix or mucosal biofilm from the recipient colon; followed by bacterial or FMT implantation. In alternative embodiments, the process comprises first cleansing the targeted microbiome space (including gut (optionally including stomach, intestine and/or colon), sinus, vagina, oral mucosa, tongue, stomach, skin, bladder, urethral, ureter, ear, bronchia, trachea, pharynx, lung or other microbiome space) of all or substantially most extraneous material such as stomach contents or non-adherent matrix or mucous; followed by removing all or substantially all of the firmly adherent matrix or mucosal biofilm from the mucosal colon; followed by bacterial implantation and/or transplantation. Additionally, in alternative embodiments, provided are the equipment and/or armamentarium for the delivery of various film-dissolving solutions; aqueous liquid formulations to dissolve the biofilm; and, the various formulations of fecal compositions to deliver to the bowel mucosa for engraftment, all combined in a smooth, efficient and effective process to achieve microbial implantation. In alternative embodiments, provided are equipment for the delivery of film-dissolving solutions which include new colonic washout machines or devices which can be specially adapted with endoscopic devices as provided herein and/or naso jejunal tubes as provided herein.

For each different condition to be treated (for example, irritable bowel syndrome (IBS), ulcerative colitis (UC), Crohn's disease (CD), autism, or constipation or other colonic diseases and conditions), first the type of equipment to be used is chosen, then a film-dissolving composition is chosen and administered, then a microbiota implantation composition (for example, FMT or other appropriate bacteria mixes) are chosen and administered, then frequency and duration of treatment is determined and administered.

In alternative embodiments, provided herein are compositions and methods that overcome or at least address (or ameliorate) the lack of implantation or engraftment after a microbial transplantation, e.g., a fecal microbiota transplantation (FMT), or the sequelae or side effects occurring after an unsuccessful transplantation (e.g., FMT) procedure. One aspect provided herein uses formulations and/or products of manufacture as provided herein for the washing out of the in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue), e.g., a colon or bowel, comprise washing out of not only the non-adherent matrix or mucous, debris or stool content but also a complete, or substantially complete, removal (washing out of) the firmly adherent biofilm which can harbor pathogenic flora (e.g., bacteria) and/or can prevent the engraftment of any incoming (infused) flora (for example, as when administering microbial transplantation, e.g., a an FMT), the flora can be from a human donor. Thus, in alternative embodiments provided herein are formulations and products of manufacture for delivering a biofilm-dissolving composition into a microbiome space in situ, for example, the colon, that can substantially (for example, remove up to about 80%, 85%, 90%, 95% or 99% or more of adherent matrix or biofilm) or completely (100%) remove mucosal adherent matrix or biofilm.

In alternative embodiments, provided herein are colonic washout equipment or devices (for example, as machines for colon lavages) to successfully achieve the implantation or engraftment of the colon with FMT material, and also having the ability initially (as a pre-FMT implantation step) to remove all or substantially most of a colonic biofilm; in some aspects these devices or products of manufacture use formulations as provided herein, for example, they comprise equipment for storing and infusing biofilm-dissolving compositions, e.g., the biofilm-dissolving compositions as provided herein.

In alternative embodiments, provided herein are methods for removing substantially (for example, removing up to about 80%, 85%, 90%, 95% or 99%) or completely (100%) removing mucosal adherent matrix or biofilm from a microbiome space in situ (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue), for example, the GI tract, or other microbiome space or microbiome-comprising tissue space or subsections thereof. In alternative embodiments, a biofilm-removing product as provided herein is infused into the patient's microbiome space in situ, for example, gut, e.g., colon, or other microbiome space or microbiome-comprising tissue space or subsections thereof, after one or more initial washouts, e.g., colonic washout(s), of the majority of the non-adherent matrix or mucous, debris or stool, for example, water or saline or a super-oxidized aqueous solution is used to wash out 90% or more of the colonic stool content.

In alternative embodiments, the process begins with the patient connected to a ‘colon washing device’, for example, a colon washing device as provided herein, where all or substantially most of the stool is removed by washing out with e.g., water or saline, followed by the removal of the biofilm using a colonic wash-out formulations, for example, a biofilm washout formulation as provided herein, which is added into receptacles or delivery modules in the colonic machine washing-out system or device.

After the initial washout, and after the subsequent infusion of biofilm washout formulation, various compositions of FMT materials are introduced into the colon. The amount and nature of the FMT formulation depends upon the illness that is being treated and the availability of the FMT materials.

In alternative embodiments, methods as provided herein further comprise use of stains or dyes in vivo to mark the presence of the biofilm in situ. The stain or dye can be administered to the individual in need (e.g., a patient) before the procedure (for example between about 30 to 60 minutes (min) before), or with the initial water, super-oxidized aqueous solution or saline washout solution, and/or with or after administration of a biofilm washout formulation, for example, administered with or in a formulation as provided herein. In alternative embodiments, dyes that can be used with methods or formulations as provided herein comprise: Coomassie Brilliant Blue, fluorescein (optionally, intravenously (IV) administered fluorescein), triarylmethane dye, rhodamine, or erythrosine B. In alternative embodiments, these stains or dyes are administered as an enema or by tablets prior to arrival of the patient in the clinic. In alternative embodiments, sufficient dye is administered such that the entire biofilm is stained blue; this permits the practitioner to see whether the removal of the film has been completed (e.g., to check the extent that the biofilm has been washed away from the colonic mucosa); when biofilm is removed from the mucosa (or washed out) the blue coloring or staining of the mucosa is depleted, and the color on the observation window of the colonic device can be measured or is seen to become water-colored. When observation confirms the all or substantially most of the dye or stain has been removed, then FMT material is infused for implantation.

### Formulations

In alternative embodiments, formulations and compositions as provided herein are liquids, or are powders or lyophilates capable of being reconstituted as a liquid formulation, which are formulated for administration to a gut or lumen, for example, for administration to a colon, to wash or lavage the gut and remove substantially all or all of the biofilm that is adherent to the gut or lumen mucosa. In alternative embodiments, formulations and compositions as provided herein are made in good manufacturing practice facilities (GMP).

In alternative embodiments, liquid formulations as provided herein (including liquids as provided herein, or powders or lyophilates as provided herein reconstituted into an aqueous saline, super-oxidized aqueous solution or water solution, wherein optionally the water can be tap water, distilled water, pure water, ozonated water, hydrogenated water, alkaline or alkalized water or any mixture thereof) comprise a soap such as a castile soap or equivalent or an IVORY™ soap or equivalent, or a baby shampoo such as Johnson's baby shampoo, wherein optionally the soap is between about 1% to 95%, 5% to 90%, 10% to 80%, 15% to 70%, 20% to 60%, or 30% to 50%, by weight or volume of the total liquid (e.g., saline, super-oxidized aqueous solution or water) formulation. For long-standing, ingrained biofilm infections the concentration of soap in an exemplary formulation as provided herein can be increased by between about 10% to about 500%.

For example, a castile soap or equivalent used in a liquid formulation as provided herein can comprise an oils, such as one or more vegetable or plant-extracted oils such as a coconut oil, olive oil, hemp oil, laurel oil, and/or jojoba oil, and an alkali (e.g., either potassium hydroxide or sodium hydroxide), diluted in water, usually distilled water; for example an exemplary formulation is:
24 oz weight (680 grams) olive oil.
16 oz weight (454 grams) coconut oil.
9.35 oz weight (265 grams) potassium hydroxide lye flakes.
32 oz (4 cups|907 grams) distilled water, for a lye-solution.
10 to 12 cups or 8 to 14 cups, distilled water to dilute.

In alternative embodiments, the IVORY™ soap or equivalent comprises or the soap formulation further comprises: a baby shampoo such as Johnson's baby shampoo, sodium tallowate, sodium cocoate or sodium palm kernelate, water, sodium chloride, sodium silicate, and magnesium sulfate; or, sodium tallowate and/or sodium palmate, water, sodium cocoate or sodium palm kernelate, glycerin, sodium chloride, and optionally one or more of the following: coconut acid, palm kernel acid, tallow acid or palmitic acid, and tetrasodium EDTA.

In alternative embodiments, soap and water formulations as provided herein, e.g., formulated for use in enemas, can comprise soap, e.g., between about ¼ to 2 ounces (oz), or between about ⅛ to 3 oz, of soap, for example, castile or ivory soap, dissolved in 1 to 3 quarts, or in about 2 quarts, of a water such as sterile water.

Because solid soap is more difficult to handle, in alternative embodiments liquid soaps are used in formulations as provided herein, e.g., and optionally the liquid soaps can be initially stored or transported in a sachet (e.g., a plastic sachet), a dispenser or a small glass or plastic container, so that it can be added to the enema solution. Because colored soaps may cause allergic reactions, in alternative embodiments non-colored soaps are used.

### Biofilm Disrupting and Anti-Biofilm Agents and Compounds

In alternative embodiments, formulations as provided herein further comprise (additional) biofilm dissolving or anti-biofilm agent or reagents or compounds, or other agents or compositions, for example, therapeutic compositions, or methods as provided herein can further comprise administration of biofilm dissolving or anti-biofilm agents or formulations as provided herein, whether or not the biofilm dissolving or anti-biofilm agent is contained within a formulation as provided herein, whether or not the biofilm dissolving agent is contained within a product of manufacture or device as provided herein; for example, the biofilm dissolving or anti-biofilm agent can be administered as a pretreatment, e.g., as or in a tablet, capsule or liquid formulation.

In alternative embodiments, in practicing the methods or uses as provided herein, biofilm disrupting or anti-biofilm compounds are administered before or during (co-administered), or co-formulated with (e.g., in a liquid enema), or separately formulated, as an administered formulation as provided herein. In alternative embodiments, disrupting biofilms are used to separate from GI mucosa, including colonic mucosa, the adherent polysaccharide/DNA-containing layer, the so-called “biofilm”.

In alternative embodiments, biofilm disrupting or anti-biofilm components or agents are administered before, during (for example, concurrent with) and/or after the administration of a formulation as provided herein, e.g., including lozenges, dissolvable wafers, strip or patches, lollies (e.g., lollypops, “pops” or suckers), candies, gums (e.g., chewing gums), which can release active compounds in the gut, aerosols, powders and sprays. In alternative embodiments, biofilm disrupting or anti-biofilm agents are administered either before treatment and/or during and/or after treatment with a therapeutic combination or composition as provided herein. In alternative embodiments, biofilm disrupting or anti-biofilm agents are used singly or in combination.

In alternative embodiments, biofilm disrupting or anti-biofilm agents comprise one or more enzymes such as a DNase, a proteinase such as proteinase K, an amylase, a lipase, a deoxyribonuclease (DNase) such as dornase alpha, or PULMOZYME™, an alginase, a lyase, trypsin, glycylglycine endopeptidase, lysostaphin, a SAL-2 enzyme from a S. aureus bacteriophage SAP-2, or a glycoside hydrolase such as dispersin B. DNases are effective in disrupting a biofilm matrix because some 30% of the biofilm is made up of DNA.

In alternative embodiments, biofilm disrupting or anti-biofilm agents comprise a super-oxidized solution (SOS) (also known as anolyte solution, or oxidative potential water) (optionally as MICRODACYN™ or MICROCYN™), optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used

In alternative embodiments, biofilm disrupting or anti-biofilm agents comprise an ozonated water, optionally used alone as a biofilm-removing or biofilm-disrupting liquid, and optionally is administered using methods described in (xiv) for super-oxidized solutions (SOS).

In alternative embodiments, biofilm disrupting or anti-biofilm agents comprise an ozone gas (which may damage a biofilm and/or its resident organisms, and optionally is administered as an insulated gas, optionally administered via a colonoscope or equivalent or by using gas bags or equivalent.

In alternative embodiments, biofilm disrupting or anti-biofilm agents that can be administered with formulations as provided herein either as components of the formulation or administered separately, including for example, biofilm disrupting or anti-biofilm agents can comprise, or be administered with, one or more antibiotics such as: triclosan, azithromycin, clarithromycin, gentamicin, vancomycin, rifaximin, rifabutin, rifampicin, nitroimidazole, streptomycin, erythromycin, roxithromycin, DEA-CP, bismuth thiols, bismuth subcitrate; bismuth subsalicylate; bismuth ethanondiothols, bismuth dimercaprol, bismuth dimercapropranol and other antibiotics, and combinations thereof.

In alternative embodiments, biofilm disrupting or anti-biofilm agents that are administered with formulations as provided herein or added to formulations as provided herein include anti-parasite antimicrobial agents.

In alternative embodiments, these biofilm disrupting or anti-biofilm agents are combined in dual, three-agent, or four or more agent combinations. In one embodiment, the antibiotic combination comprises: secnidazole, nitazoxanide and furazolidone. In one embodiment, the antibiotic combination comprises: nitroimidazoles, paromomycin, iodoquinol, doxycycline, norfloxacin, ciprofloxacin or levofloxacin, vancomycin, rifaximin, streptomycin or neomycin or any combination thereof. In one embodiment, the anti-biofilm combination comprises triclosan and dispersin B.

In alternative embodiments, other biofilm degrading or anti-biofilm substances are used to practice formulations and methods as provided herein, include or comprise: a 2-amino-imidazole such as oroidin, 2-amino-imidazole/triazole (2-AIT), or ageliferin, a fatty acid such as cis-2-decenoic acid (C2DA), S-Nitrosoglutathione (GSNO), S-Nitroso-N-acetylpenicillamine (SNAP), Gc protein-derived macrophage activating factor (GcMAF), Acyldepsipeptide or cyclic acyldepsipeptide (ADEP), DEA NONOate-Cephalosporin Prodrug (DEACP), N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7 (see e.g., Kharidia (2011) J. Microbiol. 49(4):663-8, Epub 2011 Sep. 2), nitric oxide, cys-2-decenoic acid, sodium nitroprusside, s-nitroso-l-glutathione (GSNfaO), s-nitroso-N-acetylpenicillamine (SNAP), chlorhexidine, a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or an ionic silver, a probiotic (e.g., Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, chlorine dioxide, nitrofurantoin ((E)-1-[(5-nitro-2-furyl)methylideneamino]imidazolidine-2,4-dione), zaragozic acid, norspermidine, AA-861, trehalase, parthenolide, rhamnolipid, lipoic acid, kojic acid, picolinic acid, an alcohol such as ethanol, hydrogen peroxide, hydrochloric acid, formaldehyde or luminal formalin in low concentrations, ozonated water, hydrogenated water, super-oxidized aqueous solution, nitrofurantoin (e.g., MACROBID™), hexamine hippurate (e.g., HIPREX™), potassium hydroxide, mercuric chloride, iodine or povidone-iodine (PI) (or WOKADINE™, PYODINE™ BETADINE™), and/or disodium EDTA. Ozone insufflations can also be used to disrupt the biofilm. In one embodiment, a combination of selenium and gentamicin is used to dissolve a biofilm.

In alternative embodiments, biofilm degrading or anti-biofilm substances are used to practice formulations and methods as provided herein comprise: a polyol, including xylitol, sorbitol, mannitol, erythritol, isomalt, maltitol syrup, lactitol, hydrogenated starch hydrosylates or combinations thereof.

In alternative embodiments, biofilm degrading or anti-biofilm substances are used to practice formulations and methods as provided herein comprise a surfactant, e.g., a biosurfactant, e.g., a biosurfactant extracted from a probiotic such as a Bacillus strains, including Bacillus licheniformis.

In alternative embodiments, biofilm degrading or anti-biofilm substances are used to practice formulations and methods as provided herein comprise a bacteriophage (phage), e.g., a phage directed against a bacterium found in a biofilm.

In alternative embodiments, a collection of maggots in a tea bag or bio-bag (e.g., as manufactured by Monarch Laboratories, Irvine Calif.) are used with methods as provided herein to remove or disrupt the biofilm from wounds. Maggot secretions pass through the bio-bag or teabag walls and can disrupt and dissolve biofilms.

In alternative embodiments, anti-quorum sensing (QS) compounds and/or enzymes are used as biofilm disrupting or anti-biofilm components or agents, e.g., to block several cascading pathways of the resident microbes within the biofilm. Anti-QS compounds and/or enzymes that can be used or incorporated in formulations as provided herein include: hammelitannin (HAM), a non-peptide analog of the quorum-sensing inhibitor RNAIII-inhibiting peptide (RIP); S-adenosylhomocysteine, sinefungin, a N-sulfonyl homoserine lactone and synthetic derivatives, as well as ‘biofilm-eating’ probiotics working on the QS mechanism. Biofilm disrupting or anti-biofilm probiotics that can be used or incorporated in formulations as provided herein include various Bacillus strains which secrete AiiA enzyme. In alternative embodiments, HAM and vancomycin or clindamycin are administered or incorporated into a formulation as provided herein, and they can act synergistically to increase the efficacy of antibiotics against biofilm-related infections. In alternative embodiments, RIP is used with ciprofloxacin, imipenem, and/or vancomycin, and RIP can enhance their effect.

In alternative embodiments, polymethylmethacrylate (PMMA) beads loaded with a biofilm-disrupting or an anti-biofilm component or agent, such as RIP or HAM, is administered or incorporated into a formulation as provided herein.

In alternative embodiments, a biofilm-disrupting or an anti-biofilm component or agent used or incorporated in formulations as provided herein include antibodies directed against biofilms such as, e.g., an anti-Staphylococcus aureus vaccine.

In alternative embodiments, a biofilm-disrupting or an anti-biofilm component or agent used or incorporated in formulations as provided herein include natural products or compounds, e.g., plant derived compounds, for example, ellagic acid or ellagic acid derivatives, tea-tree oils, cinnamaldehyde, chelerythrine, sanguinarine, dihydroxybenzofuran and/or proanthocyanidin.

Prebiotics that can be used or incorporated in formulations as provided herein include prebiotics from food; e.g., prebiotics can be used to pre-treat patients in methods as provided herein. Prebiotics that can be used or incorporated in formulations as provided herein include: inulin, a chicory extract, a fructan-comprising dietary fiber, N-acetyl glucosamine (NAG), an apple extract such as apple pectin, peas, tomato, rice and garlic or extracts thereof, where in alternative embodiments the prebiotics comprise substances affecting the QS.

In alternative embodiments, biofilm degrading or anti-biofilm substances are used to practice formulations and methods as provided herein comprise epigallocatechin gallate (EGCG) from green tea.

In alternative embodiments, biofilm degrading or anti-biofilm substances are used to practice formulations and methods as provided herein comprise Acetaminophen/N-acetyl-para-aminophenol (APAP)

In alternative embodiments, biofilm degrading or anti-biofilm substances are used to practice formulations and methods as provided herein comprise Aspirin (acetylsalicylic acid)

In alternative embodiments, biofilm degrading or anti-biofilm substances are used to practice formulations and methods as provided herein comprise other NSAIDs (celecoxib, diclofenac, ibuprofen, salicylic acid, etodolac, meloxicam, piroxicam, ketoprofen, naproxen, oxaprozin, indomethacin).

### Methods of Administration

In alternative embodiments, provided are methods for the treatment, amelioration (including decreasing the symptoms of, or decreasing the severity of, or inhibiting progression of) or prevention of a disease, an infection or a condition or a disease or condition caused by, initiated by or exacerbated by a pathological or abnormal microbiome, e.g., a pathological GI or colonic microbiome, for example by a pathological microbial organism such as a bacteria, bacteriophage, fungi, Archaea or virus residing or being housed by a pathological or abnormal microbiome, e.g., a gut biofilm, where the methods comprise administration to an individual in need thereof, e.g., a human or animal, a product of manufacture such as a formulation as provided herein.

In alternative embodiments, the disease or condition caused by, initiated by or exacerbated by a pathological GI or colonic microbiome is irritable bowel syndrome, chronic abdominal pain of unknown origin, autism, Crohn's disease (CD) and/or ulcerative colitis (UC). In alternative embodiments, the infection is caused by a bacterium of the genus Clostridioides, for example, C. difficile.

In alternative embodiments, one, two, three or more applications, washes or lavages of formulations as provided herein (including liquids or formulations as provided herein, or powders or lyophilates as provided herein reconstituted into an aqueous solution) are needed to remove substantially all or all of the biofilm that is adherent to the in situ microbiome space (wherein optionally the in situ microbiome space comprises a gut (gastrointestinal tract) or colon, sinus, vaginal, oral mucosa, tongue, stomach, skin, bladder, urethral, ureter, ear, bronchial, trachea, pharynx, lung, sinuses, lung or other microbiome space or microbiome-comprising tissue), e.g., adherent to the gut or lumen mucosa. In alternative embodiments, one or more applications, washes or lavages of formulations as provided herein can remove between about 70% to 100%, 80% to 99.9%, or 85% to 99%, or 90% to 98%, or about 90%, 91%, 92%, 93%, 94%, 95%, 96%, 97%, 98% or 99% or more of the biofilm adherent to the in situ microbiome mucosa, e.g., adherent to a wall or lumen of a gut, e.g., the wall of a colon.

In alternative embodiments, between about 200 ml to about 1 to 3 liters (L) of formulations as provided herein are used, or infused, per infusion event in the individual in need thereof. An individual may need only one treatment, which may include one, two or three or more infusions. Alternatively, an individual in need thereof may need multiple treatments, for example, two, three or more treatments in one day, or two, three or more treatments in one week, or treatments weekly, biweekly or monthly, after a first treatment until the disease, condition or infection is effectively treated or ameliorated, or treatments may need to be over a span of years, for example, to prevent onset or recurrence of the disease, condition or infection.

In alternative embodiments, methods as provided herein are used to substantially or completely remove both the outer biofilm layer and the more firmly adherent matrix or mucous gel, or biofilm layer, where one, two or three or more infusions or treatments may be needed. As a guideline, the loosely adherent matrix or mucus layer secreted by goblet cells appears to be much thicker in the right colon and ileum, generally becomes thinner as towards the duodenum aborally (further from the mouth, closer to the anus). In alternative embodiments, sufficient formulation as provided herein is administered to substantially or completely remove the biofilm of all or part of a GI tract, including a colon or a rectum, including both the outer biofilm later and the more mucosa-adherent matrix or mucous or biofilm layer, and alternatively, also removing one or several layers of gut lining (gut mucosa) cells. Because the thickness of a GI, or colonic, biofilm may be ‘no thickness at all’, or the biofilm may have a thickness of between about 10 to 20 micrometers (μm), a thickness of up to 1000 μm lining the tissues (e.g., human tissue) of the mucous layer. Hence the amount of formulation as provided herein, the composition of the product as provided herein (e.g., the concentration of oils or soaps used in the formulation) used in a particular procedure, or the number of treatments or infusions needed, depend on: the extensiveness of the biofilm in the colon, the depth or thickness of the biofilm, the nature (e.g., viscosity) of the biofilm, what microorganisms (e.g., bacterium, virus, bacteriophage) are embedded in the biofilm, or the nature of the disease, condition or infection which is being treated. In alternative embodiments, the amount of formulation as provided herein, the composition of the product as provided herein used in a particular procedure, or the number of treatments or infusions needed, also depend on: the amount of stirring or agitating of the luminal liquid in situ to dissolve the film, tipping of the patient head up or head down to facilitate movement of intracolonic fluids, duration of washing, and/or use of massaging of the abdomen over the area of the bowel or colon.

In alternative embodiments, methods as provided herein can have varying durations of treatment, for example, a duration of a water enema or wash, or a treatment infusion, application, wash or lavage comprising a formulation as provided herein, can vary from between 5 to 10 minutes and 10 hours, for example, between about 1 to 2 hours or 30 to 90 minutes.

In alternative embodiments, methods as provided herein, to achieve a desired washing out of a GI biofilm (for example, a complete washing out of a colonic biofilm), comprises agitating and/or stirring or mixing the contents of the bowl (including an infused formulation as provided herein) not only to remove as much of the in situ retained stool but also to reach the matrix-enclosed microbial (e.g., bacterial) populations, including the microbial organisms embedded in the matrix- or mucosa-adherent layer of the gut biofilm. In alternative embodiments, the agitating and/or stirring or mixing the contents of the bowl also facilitates or ensures damaging and/or removing the adherent matrix or mucus (biofilm) layer, optionally also including some epithelial cells in the colon mucosa to cover the depth of the pits. In alternative embodiments, the agitating and/or stirring or mixing comprises massaging the gut of the patient, or otherwise moving or shaking the patient.

In alternative embodiments, methods as provided herein comprise use of alternating soap and water washing sessions. For example, first a wash (or enema) using a formulation as provided herein (a soap wash) is administered, following by a water (e.g., sterile, tap, activated or distilled water), super-oxidized aqueous solution or saline (e.g., phosphate buffered saline, or PBS) wash (or enema). In alternative embodiments, the water wash or enema is administered first, followed by the wash or enema using a formulation as provided herein. In alternative embodiments, one, two, three or more cycles of formulation and water or saline enemas or washes are administered. Starting and/or completing the biofilm disruption and/or anti-biofilm action administrations comprising soap and water solutions (including formulations a provided herein) using a pure or distilled water, or any hypotonic solution, e.g., a solution or water having no electrolytes, allows bursting of bacterial cells, e.g., any remaining bacterial cells, by an osmotic process, where the hypotonic water enters the cells by osmosis and causes swelling of the bacteria, thus bursting the cells.

In alternative embodiments, methods as provided herein comprise either, or both, administration of formulations as provided herein from the anal end of the colon, for example, by using a colonoscope or by administration of enema, and/or from the upper gastrointestinal (GI) tract using the small bowel, for example, using a long nasojejunal tube to deliver biofilm-dissolving or biofilm-disrupting products and formulations as provided herein, and optionally afterwards, fecal microbiota transplantation, or FMT delivery, as discussed below. In alternative embodiments, a naso-gastric tube is used for the final mode of delivery, e.g., for the last infusion, e.g., of a formulation as provided herein, or a water or hypotonic solutions, or for an FMT infusion. In alternative embodiments, formulations as provided herein are infused via a washout machine, an endoscopic and/or a jejunal route.

In alternative embodiments, in methods as provided herein, biofilm disrupting or anti-biofilm agents, e.g., such as antibiotics, enzymes, probiotics, prebiotics and/or other chemicals or drugs, are administered singly or in combinations, either separately or as a component of a formulation as provided herein. Biofilm disrupting or anti-biofilm agents can be administered before, during or after water removal, or washing out, of the stool or GI contents using methods as provided herein. In alternative embodiments, in methods as provided herein, biofilm disrupting or anti-biofilm agents are administered as a pre-treatment, e.g., before any administration of washes and/or formulations as provided herein.

In alternative embodiments, biofilm disrupting or anti-biofilm agents are administered via a colonic washout machine as the first therapeutic agent to remove the biofilm followed by another therapeutic agent, a formulation of the invention comprising a soap and water. In alternative embodiments, at the end of the water or soap-and-water removal of the film, the patient empties the bowel.

### Armamentarium

In alternative embodiments, methods as provided herein are practiced using any medical armamentarium for practicing colonoscopies or nasopharyngeal endoscopies, for example, including use of known colonic washout machines, beds or chairs or colonic washout machines, beds or chairs as provided herein.

Also provided herein are novel colonic washout machines, beds or chairs; novel colonic pants or shorts; and novel rectal speculum devices, or infusion or rectal aspiration tubes. In alternative embodiments, the colonic washout machine as provided herein, which may also comprise a pump (as described below), is attached to a wall; or can be a free-standing machine or device, e.g., is mounted on rollers; or, can be attached to or be part of the colonic washout machines, beds or chairs as provided herein.

In alternative embodiments, provided is a colonic treatment system comprising: a tiltable bed or chair, with a separate or attached colonic washout machine or device having tubing connected to a speculum, optionally a speculum as provided herein, which during a procedure is inserted into the patient's anus on the tilting bed or chair. In alternative embodiments, a colonic treatment system comprises a fixed bed or chair with a speculum positioned to fit into the anus of an individual positioned (e.g., sitting or laying) on the bed or chair; both the speculum and the catchment area or opening can be co-located.

In alternative embodiments, the colonic washout machine or device as provided herein further comprises:

(a) an auxiliary container or containers capable of holding a liquid or formulation (e.g., a formulation as provided herein, or an FMT formulation) attached to the colonic washout machine or device (optionally removably attached) and operatively attached or connected to the insertion into the rectum, and optionally the auxiliary container or containers are operatively attached or connected to the rectal tube by use of a second or third, or additional, tube or equivalent connection,

and optionally the auxiliary container or containers further comprise a valve or valves disposed between the auxiliary container or containers and the rectal speculum or catheter, wherein the value or valves are capable of controlling the rate of flow of liquid or formulation (and may be pump-assisted) from the auxiliary container or containers to the rectal speculum or catheter, or the value or valves are capable of turning on or off the flow of liquid or formulation from the auxiliary container or containers to the rectal speculum or catheter, and optionally the value or valves are controlled manually, or electronically by being operatively connected to an electronic switch,

and optionally the colonic washout machine or device further comprises a pump operably connected to the rectal tube, the auxiliary container or containers, and/or the second or additional tube to move, facilitate or regulate the flow of liquid or formulation contents of the auxiliary container or containers out to a liquid or formulation flowing through the rectal speculum or catheter,

and optionally the colonic washout machine or device further comprises a heater or heating unit to heat the liquid or formulation in the auxiliary container or containers, e.g., to about body temperature,

and optionally the auxiliary container or containers or the second or additional tube or tubes comprise a filter or filters capable of separating or straining particulate matter from a liquid or formulation before it is infused into the colon;

(b) a master container or containers capable of holding liquids or formulations for infusion into a colon, wherein optionally the master container or containers are operatively connected to the auxiliary container or containers of (a), and the master container or containers are operatively attached or connected to a rectal speculum or catheter designed for insertion into a colon, and optionally the master container or containers are operatively attached or connected to the rectal speculum or catheter by use of a third tube,

and optionally the master container or containers further comprise a valve or valves disposed between the master container or containers and the rectal speculum or catheter, wherein the valve or valves are capable of controlling the rate of flow of liquid or formulation from the master container or containers to the rectal speculum or catheter, or the value or valves are capable of turning on or off the flow of liquid or formulation from the master container or containers to the rectal speculum or catheter,

and optionally the colonic washout machine or device further comprises a pump operably connected to the rectal speculum or catheter, the master container or containers, or the third tube to move or facilitate the flow of liquid or formulation contents of the master container or containers out to a liquid or formulation flowing through the rectal speculum or catheter to the colon,

and optionally the colonic washout machine or device further comprises a heater or heating unit to heat the liquid or formulation in the master container or containers to about body temperature, and a pump to assist flow of the liquid or formulation,

and optionally the master container or containers or the third tube comprise a filter or filters capable of separating or straining particulate matter from a liquid or formulation before it is infused into the colon;

(c) a pump operably connected to the rectal speculum or catheter, the master container or containers, the third tube, the auxiliary container or containers and/or the second tube, where the pump when operational (turned on) moves or facilitates the flow of liquid or formulation contents of the rectal speculum or catheter, the master container or containers, the third tube, the auxiliary container or containers and/or the second tube, to the colon,

and optionally the pump is a low pressure pump and/or a pressure adjustable pump, and optionally the pump further comprises a pressure gauge and pressure readings from the pump pressure gauge are transmitted to or are displayed to a reading device or screen on the colonic washout machine or device or remotely to a computer or a portable device, and optionally the portable device is a hand-held device or a smart phone, and optionally the pump is controlled remotely by use of a computer or a portable device or a foot pedal,

and optionally the pump is capable of providing a pulsating movement of liquid or formulation through the rectal speculum or catheter into the colon, and optionally the pulsating movement of the pump is of low or very high frequency to disrupt adherent bowel stool, and is controlled remotely by use of a computer or a portable device or a foot pedal,

(d) a first set of motor or motors operably connected to rollers or equivalent over the abdomen for massaging, shaking or vibrating an individual lying on or sitting in the colonic washout machine or device,

and optionally the first set of motor or motors are controlled remotely by use of a computer or a portable device or a foot pedal,

(f) a second set of motor or motors operably connected to the colonic washout machine or device and capable of moving the colonic washout machine or device in a tipping or side to side movement,

and optionally the second set of motor or motors are controlled remotely by use of a computer or a portable device or a foot pedal; or

(g) any combination of (a) to (f).

In alternative embodiments, provided are rectal infusion or rectal aspiration tubes comprising a first end for inserting into the colon of an individual and a second end comprising a fitting for connection to a container or source of liquid or formulation for infusion into the colon, wherein the first end of the rectal infusion or rectal aspiration tube comprises a single large opening at the and a plurality of exit holes, orifices or openings to allow passages of fluids (for example, every ½-1 cm) from the open end, for example, for a distance of between about 3 to 4 cm,

wherein the plurality of exit holes, orifices or openings are between about, or average from between about, 2 mm to 20 cm.

In alternative embodiments, provided are pairs of pants or equivalent apparel. These are worn by the patient to ‘feel clothed’, but particularly for the pants to act as a method of holding a speculum, or rectal infusion or rectal aspiration tube, in place and not have it falling out of the rectum. So this apparel is for use with a colonic washout machine, bed or chair, wherein the pair of pants comprises: an orifice or opening sufficient to allow passage of the speculum tube (or rectal infusion or rectal aspiration tube), and a clip, clamp, adhesive or stretchable material (for example, braces made of fine rope, rubber rope or similar material) capable of securing the tube (for example, a speculum tube, a rectal infusion or a rectal aspiration tube) to the patient's hips or pants such that the tube does not slip out of or through the anus. In alternative embodiments, the pair of pants is sufficiently tight-fitting or snug such that it can hold the tube in place against a pressure or pulling force on the tube with the tube being secured to the pants with the fitting, tie, snap, clip, clamp, string, rope, or adhesive or equivalent thereof. In alternative embodiments, the pair of pants is made of, or comprises, an elastic material, wherein optionally the elastic material comprises elastane, SPANDEX™ or LYCRA™. Or similar material.

In alternative embodiments, colonic treatment systems as provided herein also comprise use of other forms of equipment such as a colonoscope or similar endoscopic equipment, which can be used, modified (for example, can comprise a widened central biopsy channel to insert an ultrasonic probe) or unmodified for insertion into the bowel, or to systematically wash out the and aspirate the stool, and also optionally to then dissolve and wash out the biofilm or adherent matrix or mucus, using water, saline or either with ultrasonic power, introduced via the lumen. In alternative embodiments, once the mucosa is free of stool and film, a microbiome (e.g., FMT formulation) is infused into the caecum, and optionally sprayed onto the mucosa, e.g., upon withdrawing the instrument from the colon. In alternative embodiments, this per-endoscopic method as provided herein can be combined with a known colonic washout technology, for example, in constipated patients, as a preparation of the per-colonoscopic removal of film and infusion of FMT material. The major advantage using the per-endoscopic method is the direct vision of the mucosa.

In alternative embodiments, colonic treatment systems as provided herein also comprise naso-jejunal tubes or similar equipment for removing the stool and the biofilm from the mucosa, and optionally then infuse in a new microbiome. In alternative embodiments. these tubes have various designs; and any naso-jejunal tube can be used; and for FMT infusions, it should have a wide-enough central tube lumen or bore to permit the viscous administration of FMT materials. In alternative embodiments, the naso-jejunal tube is inserted, permitted to move distally, confirmed by distance markers and X-ray to be in the distal jejunum or even ileum, and then is anchored by thread and tape. In alternative embodiments, a first stage comprises voluminous washing out of the small and large bowel until clear fluid is passing from the rectum; and this can be followed by biofilm-dissolving solution/s (for example, biofilm-dissolving formulations as provided herein) of adequate volumes, for example, as specified herein. In alternative embodiments, after the saline washout of the mucus-dissolving solution/s is completed, an FMT administration is commenced and continued until it appears in the rectal effluent. In alternative embodiments, a major advantage of the naso-jejunal tube and related equipment is its ability to deliver and achieve engraftment to the distal small bowel as well as the colon. Colonic pants or shorts

In alternative embodiments, provided herein is a pant-like apparel (for example, pants, shorts or equivalents) to be used with a colonic washout machine, bed or chair, including a colonic washout machine, bed or chair as provided herein. In one aspect, the pant-like apparel is a novel design for pants (or a pair of pants) or shorts for the patient to wear, where the pants or shorts comprise a fitting, tie, snap, clip, clamp or adhesive or any equivalent to hold a tube (optionally an enema tube, a colonoscopic tube or rectal tube, or a rectal speculum device or infusion or rectal aspiration tube as provided herein), in place without the need for an assistant or nurse, or the patient, to hold or secure the tube to keep it from falling out of the patient. In alternative embodiments, the pants or shorts as provided herein are tight fitting or snug. In alternative embodiments, the pants or shorts comprise an orifice, hole or slit to allow a tube, for example, a rectal tube or tube or a rectal speculum device or infusion or rectal aspiration tube as provided herein, carrying a washing formulation, e.g., a formulation as provided herein, to pass through, and optionally in the pant or shorts further comprise a fitting, tie, snap, clip, clamp or adhesive or equivalent that immobilizes or holds the tube, rectal speculum device or infusion or rectal aspiration tube as provided herein, in place. In alternative embodiments, the pants comprise, or are made of, an elastic or stretchable material, and optionally the elastic or stretchable material comprises a polyether-polyurea copolymer, an elastane, SPANDEX™ or LYCRA™.

In alternative embodiments, the pants or shorts as provided herein comprise at least 2 openings or holes at the bottom end (the end approximate to the anus) to permit the holding or stabilization of a tube or speculum, or rectal speculum device or infusion or rectal aspiration tube as provided herein (by the apparel) within the anus without having to have help given by a doctor, nurse or assistant holding the speculum, tube or device in, or without having to have help by the patients themselves. In alternative embodiments, these pants or shorts as provided herein not only free the practitioner (e.g., doctor, assistant or nurse) to concentrate on the colonic washout procedure, but keep the patient from being embarrassed by having a practitioner's hand in a private area of the body. In alternative embodiments, near or approximate to each opening are hooks, snaps, adhesives or equivalent for holding the tube, device or speculum and/or the patient-inserted tube in place. In alternative embodiments, speculum devices as provided herein comprise adhesives, snaps, hooks or fittings and the like complementary to the hooks, snaps, adhesives or equivalents on the pants or shorts as provided herein.

### Colonic Washout Machines or Devices

In alternative embodiments, colonic washout machines, devices, beds or chairs are used in practicing methods as provided herein or for administering formulations as provided herein. Such machines are used to wash out fecal microbiota (the stool) from a patient that is about to undergo colonoscopy, optionally followed by FMT. This avoids needing an oral bowel preparation (prep) (or makes the oral prep optional), which often has bad taste and causes many side effects, for example, low-sodium, hypotension, nausea and vomiting.

In alternative embodiments, provided herein are new and improved armamentarium for practicing colonoscopies, including a novel colonic washout machines or devices, example, in the form of beds or chairs or equivalents, to be used as a colonic washout apparatus or device, or as a colonic washout means, for practicing colonoscopies and FMTs, including for practicing methods as provided herein or for administering formulations as provided herein.

In alternative embodiments, colonic washout machines or devices as provided herein comprise an auxiliary access, e.g., a side-access, port or intake where various liquids or formulation, including warmed liquids, including e.g., formulations as provided herein, water or saline) are added to an infusion stream, or are the infusion stream, and are infused into the bowel, for example, infused into a colon either by rectal or oral access. In alternative embodiments, the auxiliary or side-access port or intake also comprises a container or containers, and optionally a stirring device, built in to permit stirring of the infused liquid or formulation contents so as to result in a more uniform solution being infused into the GI tract, e.g., the colon.

In alternative embodiments, a warming apparatus, module or unit is provided (is included in or on a device or colonic wash device as provided herein) to heat or warm the liquids, formulations or fluids to be infused to, for example, to approximate body temperature.

In alternative embodiments, colonic washout machines or devices as provided herein are connected to an external water source, which can be a storage tank or the device can simply be connected to facility plumbing for use of tap water or other storage tank or unit; and in alternative embodiments the colonic washout machines or devices further comprise intact ports for intaking outsourced liquids or formulations such as tap water or distilled water from an external storage unit or module, and can also comprise tubes for connection to an outside water source, e.g., a tap, or a storage tank, unit or module. In alternative embodiments, a storage tank or intermediary storage or holding tank, or liquid or formulation holding module, is connected to (optionally removably connected to) or is part of a colonic washout machine or device as provided herein. In alternative embodiments, valves and readable pressure gauges can be operably connected to any intake port or tube, and in alternative embodiments, the valves are controlled remotely, for example, by use of a computer or a portable device, or a foot pedal, or combination thereof.

In alternative embodiments, colonic washout machines or devices as provided herein further comprise a pump or pumps used to facilitate and/or regulate movement or infusion of liquids or formulation, for example, formulations as provided herein, or water or saline, into the patient. In alternative embodiments, the pump is a low pressure pump and/or a pressure adjustable pump. Pressure readings from the pump can be transmitted to or be displayed to a reading device or a screen on the colonic washout machine or a device (such as a hand-held device) or readings are displayed remotely, for example, to a computer or a portable device, including a hand-held device or a smart phone. In alternative embodiments, the pump is controlled remotely, for example, by use of a computer or a portable device, or a foot pedal, or combination thereof.

In alternative embodiments, these colonic washout machines or devices as provided herein also provide (or cause) pulsation to the entering water at various adjustable frequencies, including high frequency liquid/formulation pulsing (e.g., of formulations as provided herein, water or saline), thus creating an internal or in situ vibration or shaking so as to facilitate solid content and/or biofilm dissolution and stool removal. In alternative embodiments, the pulsation and adjustable frequency controls are managed by the pump (and pump controls) operably connected to the colonic washout machines or devices as provided herein. In alternative embodiments, the pump controls are controlled by a remote device such as a hand-held device, remote device, or foot pedal, or combinations thereof.

In alternative embodiments, colonic washout machines or devices as provided herein themselves are equipped for vibration or shaking or shaking of the patient, for example, using equipment, components, motors or designs or configurations as found in a massage chair or equivalent device, for example, as described in U.S. Pat. Nos. 10,299,604; 10,285,901; 10,265,461, 10,285,89; 10,278,889; 10,231,898; 10,182,962; 10,179,084; 10,137,053; 10,034,814; 9,662,258; 9,949,618; D836,354; D848,756; D824,185. In alternative embodiments, the vibration or shaking controls are controlled by a computer or a remote device such as a hand-held device or a foot pedal.

In alternative embodiments, colonic washout machines or devices as provided herein themselves are equipped for movement, for example, comprise motors to allow the colonic washout machines or devices to be moved or adjusted, for example, if the colonic washout machines or devices as provided herein are configured as or made to be adjustable beds or adjustable chairs, then the colonic washout machines or devices can be tipped (e.g., in several directions) or reoriented, for example, they can be tipped to as much as about 35 to 45 degrees or more in any direction (for example, moving from side to side and/or moving the machine to lower or raise the legs and head) to facilitate flow and/or movement of water or formulations as provided herein in situ (infused into the patient), to ensure that the infused water or formulations as provided herein move or flow to the desired anatomic locations, for example, to ensure that the infused water or formulations as provided herein move or flow to the right colon, and/or ileum, or to the transverse colon (using e.g., side to side motion or movement of the machine). In alternative embodiments, the machine or device tilting or movements controls are controlled by a computer or a remote device such as a hand-held device or a foot pedal, or combinations thereof.

In alternative embodiments, colonic washout machines or devices as provided herein further comprise separate or auxiliary containers, modules or receptacles which are operatively connected to tubes of the colonic washout machines or devices to import liquids or formulations into the patient, for example, to fuse a liquid or formulation into the colon. The containers, modules or receptacles can be physically attached (e.g., removably attached) to the colonic washout machines or devices. In alternative embodiments, adjustable valves that can be manually or electronically controlled (e.g., opened or closed, partially or completely) are operably in line with the tubes or are operably connected to the containers or receptacles, and/or pumps, to control when and the rate at which contents of the separate containers or receptacles can enter the patient (for example, the contents of two or more liquid- or formulation-containing modules are mixed or caused to enter a tube for infusion). In alternative embodiments, separate or auxiliary containers, modules or receptacles are operably connected to a main storage tank (which itself can be separate from or part of the colonic washout machine or device) that hold formulations as provided herein, or other liquids or formulations, to be infused into the patient. Additional components such as water, saline, enzymes, antibiotics, biofilm disrupting agents, probiotics, drugs or other agents as described herein can be added to the final liquid or formulation to be infused into the patient by use of the separate or auxiliary containers or receptacles.

In alternative embodiments, pumps or gravity can control or be used to effect movement of liquids or formulations into the patient.

In alternative embodiments, if FMT components or probiotics or other materials that may comprise unwanted particulate matter are to be infused into a patient, then the separate containers, modules or receptacles, or the tubes of the colonic washout machines or devices, can be operatively fitted with filters (for example, filters of different sizes, e.g., from coarse to fine mesh) to remove or strain away unwanted particulate matter.

In alternative embodiments, colonic or colonic washout machines or devices as provided herein further comprise a pH monitor, which can be operatively connected to a main tank, a separate container, module or receptacle and or one or more of the tubes of the colonic washout machines or devices. The readings of the pH monitor can be transmitted to be displayed to a reading device or screen on the colonic washout machine or device or remotely, for example, to a computer or a portable device, including a hand-held device or a smart phone.

Any bowel or colonic washing machine, device or piece of equipment can be used to deliver a formulation as provided herein, or can be used as a starting framework to build a product of manufacture, e.g., a device or colonic washout machine, as provided herein. Bowel washing machines have been available since the 1920s, and many have the common features of supplying water from a tap, controlling the pressure of the infused water or liquids or formulation so the bowel is not distended and possibly perforated; they can also comprise a rectal infusion and aspiration speculum. For example, a HYGIEACARE® (HyGIeaCare®) Prep System (HyGIeaCare Inc., Norfolk Va.) chair can be used as a starting framework to build a product of manufacture, e.g., a device or colonic washout machine, as provided herein.

However, no available colon-washing equipment or devices to date have been built to deliver biofilm-dissolving compositions or formulations, nor have they been built to deliver FMT material products. This is because the FMT material would need to be too watery to traverse the entire colon. Thus, before development of devices and machines as provided herein, FMT had to be infused very close to the anus to bypass the entire colon-washing (washout) machine and reduce dilution by the water or saline that might be used to washout the colon.

In alternative embodiments, a bowel or colonic washing machine, device or piece of equipment as provided herein comprises or incorporates a tilting bed, chair or equivalent patient support, which optionally can be electrically driven, and optionally can be tipped upwards and downwards to more than about 35° and optionally can be controlled by a hand or foot pedal. In alternative embodiments, the tilting bed, chair or equivalent patient support component as provided herein can rotate clockwise or anti-clockwise. These movements of the tilting bed, chair or equivalent patient support can collectively help deliver or move the internal liquid or formulation (e.g., the infused liquids or formulations) inside the colon, e.g., to either the left, transverse and/or the right colon, so permitting larger amounts of liquid or formulation reaching those areas. The bed may also be equipped with a vibrating mode or cycle.

In alternative embodiments, a bowel or colonic washing machine, device or piece of equipment as provided herein comprises or incorporates one or more intake ports, cassette spaces or cavities, each which can take (or input) a cassette, cartridge, sachet or a removable cartridge housing. In alternative embodiments, at least two different types of cassettes, sachets or cartridges are used, and they can be attached (for example, clipped or clamped) into place in the intake port, cassette space or the cavity either directly, or they can be first placed in a cartridge housing and the housing is inserted into the cavity, cassette space or intake port. In alternative embodiments, a cartridge, sachet or other container comprises a formulation as provided herein, for example, a liquid or formulation, powder or lyophilate formulation for dissolving a biofilm; a drug or an active agent; or, an FMT material. In alternative embodiments, to accommodate a cassette, sachet or a cartridge that comprises or has contained therein a powder or a lyophilate formulation, the intake port or cavity is operably connected to an intake tube and an output tube such that when the cartridge, sachet or other container (or cartridge housing) is inserted into the cavity or the intake port a liquid or formulation (such as water, saline or soapy water formulation) can be caused to flow through the cassette or cartridge (and optionally the flow of the liquid or formulation into and/or through the cassette, sachet or cartridge is controlled by a valve) and the contents of the cassette, sachet or cartridge, for example, the powder or lyophilate, is carried by the liquid or formulation out of the cassette, sachet or cartridge and into the patient either directly or via connection to a colonoscope, tube or speculum. In alternative embodiments, the output tube is directly connected to a delivery device, for example, a speculum, colonoscope, tube or equivalent delivery device, such that its contents are delivered into the colon of a patient. In alternative embodiments, the cassette or cartridge output tube is directly connected to another tube that is carrying a liquid or formulation such as water or a formulation as provided herein to a delivery device, for example, a speculum, colonoscope or equivalent device, for delivery to the colon. In alternative embodiments, the cassette, sachet or cartridge output tube is connected directly or indirectly to a holding or mixing tank where it is held and/or mixed with the contents of other liquids or formulations before being delivered to a colon of a patient.

In alternative embodiments, for example, when the cassette, sachet or cartridge comprises FMT material, which may include cultured consortia, the cassette or cartridge comprises at least one filter, or at least one filter operably connected to the cartridge, and the filter is fitted or positioned in the cavity or cassette space or is fitted or positioned approximate to an output port. The filter can be fitted in a filter repository (e.g., a slot) operably connected to the cassette or cartridge output tube, the distal end or the cassette or cartridge, or the cassette or cartridge housing, and in alternative embodiments the filter(s) can be independently removed and replaced with clean filters.

In alternative embodiments, the device, e.g., the one or more intake ports, or cavities, or cassette, sachet or cartridge housings, further comprise or are operably connected to or approximate to a warming module or device, thus, a liquid or formulation in the cassette or cartridge, or a liquid or formulation flowing through the cassette or cartridge, is warmed in the cassette or cartridge. The device, e.g., the one or more intake ports, or cavities, or cassette or cartridge housings, can further comprise an thermometer or thermostat capable of reading the temperature of the liquid or formulation and/or controlling the temperature of the liquid or formulation, where the liquid or formulation can be warmed and/or maintained at approximate body temperature, for example, the liquid or formulation can be warmed and/or maintained at about 37° C. In alternative embodiments, the thermometer or thermostat is remotely read and/or is remotely controlled, for example, by a hand-held device or a computer.

In alternative embodiments, the contents of the cassette, sachet or cartridge after flowing out of the cassette or cartridge, are delivered directly to a tubing near the anus, for example, the contents are delivered to the Y division of a speculum.

In alternative embodiments, provided is a modified colonoscope, e.g., to practice methods as provided herein, or to deliver formulations as provided herein, for delivering formulations as provided herein, e.g., for delivering biofilm-dissolving agents or FMT materials to a patient. In alternative embodiments, the colonoscopes or devices as provided herein are adapted to dissolve biofilm-dissolving agents or FMT materials and them to a bowel.

In alternative embodiments, a patient takes a regular bowel preparation orally, after which the patient is colonoscoped, and the colon can be filled with a fluid or liquid or formulation such as a formulation as provided herein. With the colon full of the liquid, formulation or fluid, the colon effectively provides a wide bore luminal channel which can accept a high energy endoscopic ultrasonic device. In alternative embodiments, this device can transmit ultrasound power through the liquid or formulation (e.g., water with or without soap) to the mucosa on the walls of the bowel. This ultrasonic power can disrupt or crack the adherent matrix or mucosa-adherent biofilm and/or damage it such that it can be made more soluble and/or removable from the mucosal wall. In alternative embodiments, the liquid comprises a soap, or a soap can be added to the liquid, or the liquid comprises soap and saline or soap and water. In alternative embodiments, the liquid or formulation is agitated within the bowel first by repeated aspirating and infusing, and also optionally by massaging or vibrating the abdomen (e.g., using a device or vest as provided herein). In alternative embodiments, the ultrasonic power is applied to disrupt and/or crack the the adherent matrix or mucosa-adherent biofilm, and after which the liquid or formulation is aspirated. In alternative embodiments, there can be multiple infusions of liquid or formulation, for example, the additional infusion can comprise use of a liquid or formulation comprising antibiotics such as antiparasitic agents that have been known to break up biofilm, for example, the additional infusion can comprise use of a formulation as provided herein, which can comprise an antibiotic (for example, furazolidone, nitazoxanide and/or secnidazole), a prebiotic, a probiotic, a drug and the like. In alternative embodiments, after the wash or washings, the bowel can be aspirated with the colonoscope to the extent that it is almost or substantially empty of any fluid or liquid or formulation. In alternative embodiments, after the colonic mucosal biofilm has been substantially removed, e.g., the colon is subjected to an additional wash, for example, washed with water and/or a saline; and then afterward an FMT material is infused into the colon or caecum, optionally the FMT material is infused and/or sprayed along the entire length of the bowel. Thus, a new biofilm can be formed along the entire bowel without permitting time for the old (and possibly pathogen-contaminated) biofilm to regrow.

In alternative embodiments, a naso-jejunal tube is used, and it is inserted into the GI tract from the nose and/or mouth; advantages to using a naso-jejunal tube include orthostatic washout and no need for a colonic machine washout, and the naso jejunal tube's ability to wash out with all current colonic and/or fecal material; however possible downsides to using naso-jejunal (NJ) tubes include lack of vision of the mucosa, as is achieved by a colonoscope, and lack of the ability to use ultrasound to disrupt or crack GI tract (e.g., colonic) adherent matrix or mucosa-adherent biofilm.

### Rectal Speculum, or Rectal Infusion or Rectal Aspiration Tube

In alternative embodiments, provided herein are novel rectal speculum devices, or infusion or rectal aspiration tubes, which can be connected to any colonic washout machine or device, or a colonic washout machine or device as provided herein. In alternative embodiments, these novel speculum, or rectal infusion or rectal aspiration tubes comprise a plurality of exit orifices or holes, for example, drilled exit holes, at the end of the tube to be inserted into the patient; and also comprise smooth or polished surfaces to prevent scratching or irritation upon the tube's physical contact with mucosa. In alternative embodiments, the plurality of exit holes are between about, or average from between about, 2 mm to 20 cm. The exit holes allow water exit if the distal opening is occluded by the usually loose rectal mucosa.

Before the design of speculum devices as provided herein, designed speculums were rudimentary, addressing more the infusion aspect and less on the drainage of the colon. Because this old equipment was not designed by people who colonoscope patients (who realize that aspiration through the colonoscope causes mucosal obstruction of the aspiration orifice), previous speculum devices and their aspirating holes were neither optimally ergonomic or optimally functional. In using the older equipment, the liquid or formulation infused into the colon is unable to come out through the same speculum because the speculum becomes closed over by mucosa that covers the openings as the liquid or formulation attempts to come out driven by gravity. In some older devices, liquid or formulation pulsation can only move in the one direction—forward—due to absence of distant holes in the speculum to allow a return movement.

In alternative embodiments, speculum and devices as provided herein solve these problems, and are more ergonomic and are optimally functional: for example, by having a central opening at its distal end (the end farthest from the operator, the end furthest in the colon), and also comprising numerous (a plurality of) openings (for example, drilled or milled openings, or designed openings if the device is made by 3D printing) circumferentially close to the distal end or tip, for example, a plurality of openings at about 1, 2, and 3 cm from the distal tip, or a plurality of openings positioned between about 0.5 to 20, or 1 to 10 cm from the distal tip of the device or speculum. This positioning of openings effectively prevents (or substantially prevents) the mucosa from completely closing all exits (all of the openings), and permits infused water and dissolved stool to exit out of the colon through the device. FIG. X schematically illustrates an exemplary rectal aspiration tube or speculum as provided herein.

In alternative embodiments, an exemplary mechanism or capability built in to a colonic washout machine as provided herein is the ‘waterhammer effect’, which is created by movement of a water or liquid or formulation column for a short distance, for example, for between about 0.5 to 10 cm, or between about 1 mm to 5 cm, using a piston-driven system, a pump, or an equivalent such that the water or liquid or formulation column is induced to have a back-and-forth movement of the water in situ, for example, the induced back-and-forth movement does not distend the caecum, but is effective in washing the edges.

This ‘waterhammer effect’ could not work without the novel design of the speculum and devices as provided herein, which because of the novel orientation and placement of the distal or tip openings in the device have open exit holes at all times (as explained above, without such openings return water movement would be blunted by mucosa blocking the exit of fluids at the tip or distal end of the device).

In alternative embodiments, the cycling of the “waterhammer” capability of a device or machine as provided herein can be turned up to a very high speed, thus producing vibration only rather than actual movement of water or liquid or formulation.

### Vests

In alternative embodiments, also provided are novel vests, tops or shirts or equivalent apparel to be worn by a patient having a colonic wash, for example, for a patient have a wash comprising use of methods, formulations and/or devices or products of manufacture as provided herein. In alternative embodiments, these vests provide a massage or ripple effect on the abdomen (e.g., the lower abdomen), for example, they provide a ripple bed-type effect. In alternative embodiments, the vest or shirt is based on a design of a vest used to reduce deep venous thrombosis. In alternative embodiments, the abdominal vest as provided herein can help mechanically compress and/or move infused water around the colon, thus assisting to clean the bowel wall of stool; and if a biofilm washing agent is infused, to cause more thorough cleaning of the biofilm layer.

In alternative embodiments, vests provide a massage or ripple effect on the abdomen by use of a plurality of motors (e.g., from two to ten or more motors) implanted within the vest will can cause rollers or arms in the vest to create a massage effect on the abdomen. In alternative embodiments, the motors are operatively connected to a control device, e.g., a computer, cell phone or a handheld device, that can control the speed and intensity of the motors. The control device can be operated by the patient or an operator.

For example, the vest can comprise or incorporate components or elements for creating a massage effect on an abdomen, or a variable heat effect, as described in, e.g., U.S. Pat. No. 10,016,335 (describing an air pulse generator); U.S. Pat. Nos. 8,480,603; 8,202,235; 8,172,778; 7,785,280; 7,846,113; 7,770,479; 7,207,953 (describing a control module having a processor connected to a plurality of massage heads for controlling oscillation and direction of oscillation, each said massage head having a ball shaped free end extending from the inner side of a vest, the ball shaped free end is oscillated by a massage motor connected to an opposite end of the massage head); U.S. Pat. Nos. 7,121,80 6,551,259; 6,329,638; 6,193,678; 5,938,627; or U.S. patent application publication nos. 20190183723 and 2016015809.

In alternative embodiments, the vests further comprise one or more heating devices which also are operatively connected to a control device, e.g., a handheld device, that can control the temperature of the vest.

In alternative embodiments, the vest is attached (optionally removably attached) to the bed, chair or equivalent patient support, to cause a ripple effect on the abdomen, and can help with the compression of the abdomen segmentally, e.g., can help with manual compression of the abdomen.

### Fecal Microbiota Transplantation

In alternative embodiments, methods as provided herein further comprise administration of a fecal microbiota transplantation, or FMT. In alternative embodiments, following treatments using formulations as provided herein, or using methods as provided herein, FMT is carried out, e.g., once or twice or can be continued for a number of days or weeks until such time as a new biofilm is formed from the newly infused FMT material not containing the chronic biofilm infectious agent causing or exacerbating the original disease, illness, infection or condition.

In alternative embodiments, the FMT and the FMT procedure can comprise any FMT composition or procedure known in the art, for example, as described in U.S. Pat. Nos. 10,251,914; 10,226,431; 10,220,089; 10,064,900; 10,064,899; 10,028,980; 10,058,576; 9,623,056; 9,610,308; 9,572,842; 9,468,658; 9,408,872; 9,320,763; 9,308,226; 9,192,361.

As discussed above, in alternative embodiments, the FMT material can be housed in a cassette or cartridge which can also fit into or be operatively inserted in a colonic washing machine cassette or cartridge space, or the cassette or cartridge is first fitted into a housing, which in then inserted into a cassette or cartridge space. In alternative embodiments, the cassette or cartridge is operatively fitted with a filter at the bottom or distal end; and optionally the cassette or cartridge is operatively fitted with or approximate to a hearing module or device, and optionally a thermostat, such that the temperature of the contents of the cassette or cartridge such as a liquefied FMT material is raised or maintained at body temperature.

In alternative embodiments, liquid or formulation from the cassette or cartridge flows into a tubing that enters the colon; for example, liquid or formulation from the cassette or cartridge flows into a narrower tube that is connected to (flows into) a Y-division in the tubing just next to the anal access to the colon.

In alternative embodiments, the cartridge or cassette is the same size and shape, or is fitted, to fit snugly into a preformed compartment or cartridge or cassette compartment or housing. The cartridge or cassette also can be disposable.

In alternative embodiments, the cartridge or cassette comprises or contains a mix of various components, including for example a formulation as provided herein, to be delivered to the GI tract, for example, to be delivered to a colonic wall for implantation.

In alternative embodiments, the cartridge or cassette comprises or is fitted with a filter at the bottom or distal end, as discussed above.

In alternative embodiments, a stirring device is added to the cartridge or cassette.

In alternative embodiments, the cartridge or cassette contains an anaerobic environment, for example, the cartridge or cassette contains an anaerobic environment with FMT material.

In alternative embodiments, the cartridge or cassette comprises or contains therein stool material, for example: lyophilized stool; fresh liquefied stool; frozen stool which has been thawed out; pre-filtered stool with or without spiked (or added) bacteria, such as beneficial probiotics, for example, probiotics comprising a Faecalibacterium such as Faecalibacterium prausnitzii; and/or re-suspended, cultured or liquid lyophilized bacteria or spores. In alternative embodiments, the cartridge or cassette comprises or contains therein lyophilized stool suspended in water or saline with or without a spiked (or added) bacteria. In alternative embodiments, the cartridge or cassette comprises or contains therein ultra-filtered material, where the ultra-filtration removes substantially most of the bacteria and fungi but leaves behind viruses and bacteriophages. In alternative embodiments, the cartridge or cassette comprises or contains therein re-suspended, cultured or liquid lyophilized bacteria or spores with or without spiked (added) additional components such as drugs, probiotics or prebiotics.

In alternative embodiments, the cartridge or cassette comprises or contains therein a temperature sensitive polymer such as THERMOGEL™ or equivalent, which when mixed with a fecal microbiota such as FMT or another bacterium it is a liquid at room temperature but gels solidifies when it reaches or is near body temperature (for example, gels at about 37°).

In alternative embodiments, the total volume of the cartridge or cassette is between about 300 ml to 1000 ml, 100 ml to 2000 ml, or 50 ml to 3000 ml.

In alternative embodiments, the total volume of the cartridge or cassette moves directly into the speculum close to the anus so as to bypass large volumes of tubing within a pumping machine. Alternatively, a patient is directed to get up and empty the bowel in the bathroom; and afterwards and treatments using formulations as provided herein, or using enema bag and catheter can be used to run an FMT liquid or composition into the colon at a much lower volume such that the FMT would not have to pass through its package through the cassette.

### Tube Implantable in the Caecum

In alternative embodiments, methods as provided herein further comprise inserting an implantable catheter/tube via a colonoscope to the ceacum and clipped to the mucosa/wall to deliver biofilm dissolving chemicals in situ, and later FMT or probiotic consortia material.

This method may be used when a prolonged biofilm dissolving pre-treatment is required to treat a patient with advanced disease and to reduce the cost of treatment as less colonoscopies would be required. By anchoring the tube/catheter in the ceacum, the biofilm dissolving liquid can be released at the beginning of the colon where it is hardest to get to due to the anatomy of the large bowel. By placing the tube/catheter in situ the colon can be washed repeatedly and effectively with minimal patient discomfort.

After the colon has been washed and the biofilm dissolving agents have performed their function, the inserted tube/catheter can be used to deliver the therapeutic FMT or probiotic consortia dose to implant a new healthy microbiome and biofilm at the beginning of the large bowel and to then implant downstream as it naturally moves down along the large bowel towards the rectum. The implantable tube/catheter may at least allow for rapid and easy delivery of one or multiple treatments without the costs of multiple colonoscopies and anaesthesia. The implantable tube/catheter can be left in place for a few days to allow for follow up FMT or probiotic consortia infusions to maximise the chances of successful microbiome engraftment.

The implantable tube/catheter may have a singular or multiple openings along its path to release biofilm dissolving liquid and FMT or probiotic consortia along the length of the bowel to both fill the bowel faster with the biofilm dissolving agents and to also cover more large bowel surface area with the FMT or probiotic consortia as quickly as possible to ensure the pathogenic bacteria causing the disease state have a minimal chance of surviving to set up new colonies of pathogenic biofilm.

At the conclusion of therapy as decided by the treating physician, the implantable tube/catheter may be removed via a colonoscopy or manually withdrawn, and the patient released from care.

Any of the above aspects and embodiments can be combined with any other aspect or embodiment as disclosed here in the Summary and/or Detailed Description sections.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Exemplary Protocols—Colonic Washes

This example describes exemplary methods, and devices and formulations as provided and methods for using them.

A colonic washout machine is connected to a patient's rectum and a washout (or infusion) is carried out using a formulation as provided herein, for example, a water followed by soap and water, finishing off with water alone. The volumes may be between about 1 through to about 45 L of each of these cycles. In one exemplary protocol, the patient starts a washout using 36 L of water, then follows with 8 L of a formulation as provided herein comprising soap and water, finishing with 2 L of plain water. The patient then is allowed to empty the bowel while connected to the washout machine, rise and empty the rest of the colonic contents in the bathroom. This can be then followed by FMT or other microbiome-like suspension product infusion soon after and at 2 hour (hrs) intervals completing 1, 2, 3, 4, 5, 6 or more infusions that day to encourage best engraftment.

In one exemplary protocol, a colonic washout machine is connected to the patient's rectum, and the plain water carries one, two, three or four or more anti-parasite antibiotics, for example, in one embodiment the plain water comprises nitazoxanide, secnidazole, diloxanide furoate, and furazolidone. In one exemplary protocol, the volume of this antibiotic-comprising infusion is about 2 to 5 liters (L), or can be between about 1 to 10 L. The patient can have the liquid or formulation moved around the bowel by massage and by tilting the bed upwards and downwards to reach the entire colon and to make sure that the anti-parasite agents penetrate the mucosa and the biofilm.

The next phase is the use of soap and water containing liquid or formulation of between about 3 to 10 L and this is then slowly massaged around the abdomen to make sure the entire colon is covered with further removal or any biofilm and early removal of the 1st layer of so of mucosal cells. It is then emptied and the patient is again treated with about 8 to 10 L of plain water to remove the entire leftover soap and water and any leftover antibiotics in preparation of multiple FMT infusions as described in scenario A.

### Example 2: Exemplary Protocols—Colonic Washes

This example describes exemplary methods, and devices and formulations as provided and methods for using them.

A patient is connected to a colonic washout machine and between about 2 to 6 liters (L) of plain water are used to remove most of the colonic contents, including the stool. Next, between about 3 to 5 L of a formulation as provided herein (comprising soap and water) are used to wash out the biofilm, most likely also including a washout of the 1st layer of cells.

In the next exemplary stage, about 2 liters of water containing dissolved anti-parasite agents, for example as described above, are infused and agitated and moved around the colon making sure it reaches the right side of the colon using bed tipping and massage. This solution is then emptied.

In the next exemplary stage, between about 2 to 4 L of enzyme-containing water including an enzyme such as DNase (such as e.g., dornase alpha, or PULMOZYME™), amylase, protease, and/or lipase are combined together, optionally also with N-acetyl-cysteine or another component or components, as described above; and this is infused in the patient.

The bowel is then emptied while the patient is still attached to the washout machine. The patient is then allowed to empty the bowel in the bathroom.

Optionally, next infusions of FMT are commenced, as described in Example 2, or above. In alternative embodiments, FMT is continued the next day, and in some, over the next 3 to 5 days, or more, as needed to make sure every mucosal area of the colon is covered with fresh fecal microbiome as it recovers from any damage the infusion of formulations as provided herein, or the water fusions, may have incurred. Frequency of FMT infusions vary as needed to ensure the colon has recovered sufficiently that the new FMT material can and does takes residence in the colon, and that the colonic mucosa generates its own healthy biofilm.

In alternative embodiments, any combinations and/or permutations of methods as provided herein using formulations as provided herein to effect biofilm colonic washout and destruction protocols are utilized. In alternative embodiments, exemplary applications to remove pathogenic colonic biofilm with the goal of having the patient regenerate healthy (e.g., uninfected biofilm) in the colon are used on patients who have failed previous biofilm removal protocols.

### Example 3: Exemplary Protocols—Naso-Jejunal Tubes

This example describes exemplary methods, and devices and formulations as provided and methods for using them.

Patients are treated with long naso-jejunal tube in situ, washing out the colonic contents using formulations as provided herein from “above”, via the small bowel. In alternative embodiments, about 3 to 15 L or more of water are first infused into the patient (optionally, infused very slowly), followed by infusion of a formulation as provided herein (for example, comprising soap and water, or a composition or formulation as provided herein).

In alternative embodiments, this is followed by a further washout, or multiple washouts, with water or saline.

In alternative embodiments, this is followed by multiple infusions of about 10 cc to about 30 cc of a formulation as provided herein (e.g., multiple infusions of an IV formulation comprising metoclopramide (or PRIMPERAN™, REGLAN™) as a prokinetic, every about 1 to 6 hours (hrs), or over the next 24 to 48 hours. The doses and durations mentioned herein are adjusted for each individual patient.

### Example 4: Exemplary Protocols—FMT

This example describes exemplary methods, and devices and formulations as provided and methods for using them.

Any of the variations of formulations as provided herein are used to facilitate the success of the washing machine performance and implantation of FMT microbiota. Starting with the microbiota containing products one can use fresh homogenized donor stool which has not been frozen, and alternative FMT material that has been produced either aerobically or anaerobically.

An FMT product can be contained in a screw top plastic or glass container. The FMT product may be a homogenized uniform product that is frozen and is open as the patient arrives for the treatment.

In alternative embodiments, various preserving agents such as trehalose or glycerol, are added to the FMT.

In alternative embodiments, the FMT donor stool contains the full spectrum of microbiota, and can be filtered and contained either fresh or frozen. In alternative embodiments, the FMT material is ultra-filtered and/or lyophilized, or can be a powdered microbiota, or can be a powder comprising mixtures of agents or products as described herein.

In alternative embodiments, the FMT are lyophilized products, and can be made up of either full spectrum microbiota with added protective agents or with added fortified components such as Fecalibacterium prausnitzii spores, various Lactobacilli, Firmicutes, Bacteroidetes or other non-pathogenic agents.

In alternative embodiments, the FMT comprise fully cultured consortiums of various microbial groups, and can be mostly bacteria.

In alternative embodiments, the FMT is engrafted either into the small bowel and/or colon through a naso-jejunal tube or through the rectum into the entire colon from below.

In alternative embodiments, formulations as provided herein, or formulations for use in methods as provided herein (e.g., for use in dissolving a colonic biofilm) comprise one or more enzymes, one or more antibiotics, or one or more enzymes combined with one or more antibiotics, e.g., using enzymes and/or antibiotic as listed above.

In alternative embodiments, quorum sensing inhibitors, e.g., as listed above, are added formulations as provided herein. In alternative embodiments, patients are pre-treated with probiotics (e.g., as described herein) including for example: inulin, a chicory extract, a fructan-comprising dietary fiber, N-acetyl glucosamine (NAG), an apple extract such as apple pectin, rice tomato garlic extracts and/or peas, each in various concentrations.

In alternative embodiments, formulations as provided herein, or formulations for use in methods as provided herein are infused via an enema access and/or through a naso jejunal tube inserted into the patient to cover both the distal small bowel as well as the entire colon.

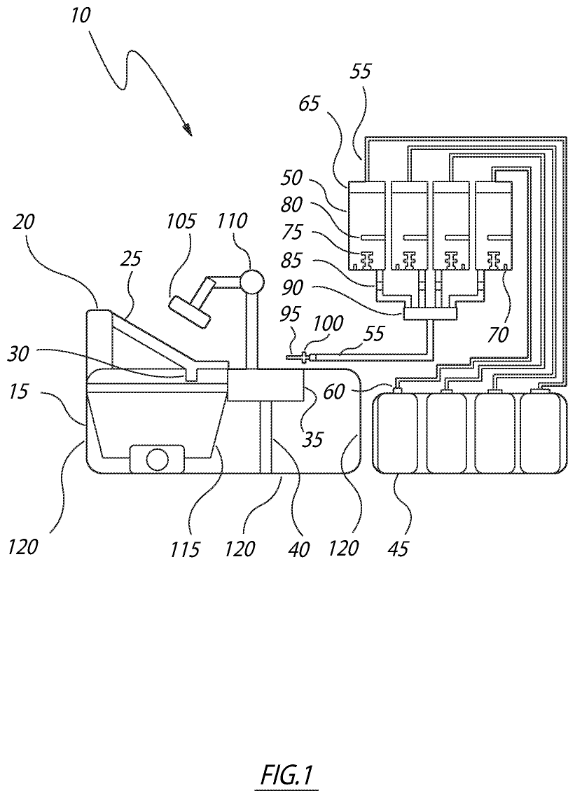

FIG. 1 schematically illustrates an exemplary colonic washout machine 10 as provided herein, the colonic washout machine 10 being of the ‘Open’ type used to carry out the initial part of the bowel cleaning and biofilm degrading and/or dissolving before a patient undergoes the colonoscopic treatment.

The colonic washout machine 10 includes a colonic bed 15 which has a solid backrest 20 and a removable bed cover 25. The patient lies on the colonic bed 15 and at least partially on the backrest 20. To ensure cleanliness and the prevention of contamination between treatments, the removable bed cover 25 is used, and may be replaced with a new cover for each patient. A recess/notch 30 is built into the body of the bed 15 where part of the removable bed cover 25 fits into, so as to prevent the bed cover 25 from sliding forward while the patient is lying thereon.

During a colonoscopic treatment, fluid that is evacuated from the patient's bowel is released into the bowl 35 and released to the sewage system via a sewage pipe 40.

A plurality of large capacity fluid reservoirs 45 containing different types of biofilm dissolving and/or degrading liquids have their contents transferred via a pump (not shown) to the fluid containers 50 via fluid lines 55. In the embodiment as shown in FIG. 1, four fluid reservoirs 45 and four corresponding fluid containers 50 are shown, with four respective fluid lines 55 providing a connection therebetween. It will be appreciated that in other embodiments (not shown), any number of fluid reservoirs 45 and fluid containers 50 may be provided, depending on the requirements of the machine 10. Each fluid line 55 links one of the large capacity reservoirs 45 to a specific or respective fluid container 50 as to minimise the mixing up of liquids used. This is also reinforced by the use of different sized reservoir caps 60 to ensure that only one type of fluid is used in each reservoir 45 supplying the fluid containers 50.

The fluid reservoirs 45 are placed on or near the ground to simplify the filling up process if done manually, as lifting of heavy weights may be involved. The fluid containers 50 can also be filled manually by removing container caps 65 in situations where there is not enough floor space available to install the large reservoirs 45. The fluid containers 50 may each contain a temperature sensor 70 arranged to detect and monitor temperatures of fluid within the container 50, so as to ensure the fluid temperature remains within a predetermined range as to not cause thermal injury to the patient. The fluid containers 50 may also each contain a fluid mixing propeller 75 which continuously or intermittently mixes the liquid in the container 50 as to keep the solution elements in the proper dilution parameters at all times, and a heating element 80 that may warm the liquid to a temperature within a predetermined safe and comfortable range for the patient.

During treatment, the fluids in the fluid containers 50 flow down the fluid lines 55, then through a fluid flow control valve 85, and enter a junction container 90 where the different treatment fluids may be mixed in a predetermined solution concentration or just allow the flow one treatment liquid at a time on its own based on the treatment protocol used. The liquid then continues its flow down the fluid line 55 (shown as a single line in FIG. 1) and exits via a flexible small diameter rectal catheter 95 that is inserted in the patient's rectum during treatment. The flexible small diameter rectal catheter 95 has an insertion flange 100 which prevents the patient, nurse or treating doctor from inserting or entering the flexible small diameter catheter 95 too far in the patient's rectum and potentially causing an injury.

To enhance the biofilm dissolving and/or degrading treatment, a vibrating plate 105 may be attached to a stand 110 that is either connected to the colonic bed 15 or a free-standing maneuverable stand is placed in contact with the patient's abdomen and turned on when the patient's bowel is either fully or partially filled with the treating fluid. The vibrating plate 105 may be run continuously or intermittently. The vibrating plate 105 may be flat, slightly concave or very concave as to contact as much as possible of the patient's abdomen area. The vibrating plate 105 may be made of a solid block material like plastic or metal or a pliable material that can conform to each patient's body shape like a vibrating mechanism embedded in a cloth like material, and may be attached as a wide belt, vibrating the patient's abdomen over the distribution of the colon.

A tilting mechanism 115 is situated under the colonic bed component 15 of the colonic washout machine 10. The operation of the tilting mechanism 115 may be controlled by the nurse or treating doctor either via a foot pedal or other control mechanism (not shown). The tilting mechanism 115 can either raise or lower either side of the bed component 15 of the colonic washout machine 10 in order to place the patient in the preferred position for the whole treatment or for a specific (partial) treatment segment. It will be appreciated that in one form, the tilting mechanism 115 is situated inside one or more removable panels of the colonic washout machine 10, for example panels 120 of the bed 15, and is not visible from the exterior during normal operation.

A number of embodiments of the invention have been described. Nevertheless, it can be understood that various modifications may be made without departing from the spirit and scope of the invention. Accordingly, other embodiments are within the scope of the following claims.

1. An aqueous liquid formulation comprising:
(a) a soap,
(b) at least one compound or composition comprising, or selected from the group consisting of:
(i) at least one biofilm disrupting agent comprising at least one enzyme;
(ii) at least one antibiotic;
(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l-glutathione (GSNfaO), s-nitroso-N-acetylpenicillamine (SNAP), chlorhexidine, iodine, povidone-iodine (P1) (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (and optionally the probiotic comprises a Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water, hydrogenated water, activated or electrolyzed water, a super-oxidized aqueous solution (optionally OXUM™, MICRODACYN™, DERMACYN™), nitrofurantoin (e.g., MACROBID™), hexamine hippurate (e.g., HIPREX™), potassium hydroxide, mercuric chloride, boric or boronic acid, disodium EDTA, a phytocannabinoid, optionally cannabidiol (CBD), an alkyl dimethylol alkanate (ADMA), or any mixture or combination thereof;
(iv) at least one polyol or a wetting agent;
(v) at least one surfactant or biosurfactant;
(vi) at least one anti-quorum sensing (QS) compound;
(vii) at least one prebiotic,
and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;
(viii) a stain and/or a dye,
wherein optionally the stain or dye comprises Coomassie Brilliant Blue, triarylmethane dye, rhodamine or erythrosine B;
(ix) a stool softening agent or a laxative,
wherein optionally the stool softening agent or laxative comprises: glycerin, sorbitol, lactulose, polyethylene glycol (PEG), a docusate, a docusate salts or a dioctyl sulfosuccinate or a mixture thereof;
(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter,
and optionally the activated carbon or charcoal is or is formulated as a powdered, granular or extruded activated carbon or charcoal, or is formulated as a bead-activated, woven or polymer-coated carbon;
(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;
(xii) pure (or substantially pure) or distilled water (H2O) (optionally alkaline water), optionally used alone as a biofilm dissolver exploiting its hypotonic nature to penetrate bacteria resulting in swelling and bursting of the bacteria or other pathogen, and optionally the pure, alkaline or distilled water is used as an enema,
and optionally the enema or a colonic washing is by infusing infused the pure, alkaline or distilled water by use of a colonic machine or equivalent, or by use of a naso-gastric (NG) long tube, or equivalent, and optionally ozone or ozonated water is administered after administration of the pure, alkaline or distilled water;
(xiii) iodine, povidone, povidone-iodine (PI), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;
(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,
and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,
and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,
and optionally water, saline, a soap and water mixture, a super-oxidized solution (SOS) (also known as anolyte solution, or oxidative potential water), is used as a dissolving or suspending liquid,
and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or equivalents of mixtures thereof,
and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;
(xv) a super-oxidized solution (SOS) (also known as anolyte solution, or oxidative potential water) (optionally as MICRODACYN™ or MICROCYN™) optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;
(xvi) an ozonated water, optionally used alone as a biofilm-removing or biofilm-disrupting liquid, and optionally is administered using methods described in (xiv) for super-oxidized solutions (SOS);
(xvii) an ozone gas (which may damage a biofilm and/or its resident organisms, and optionally is administered as an insulated gas, optionally administered via a colonoscope or equivalent or by using gas bags or equivalent,
and optionally ozone is administered during a colonoscopy, optionally either in air or with CO2 as insufflating gases,
and optionally the ozone gas is substituted or replaced by CO2, and optionally the ozone gas is aspirated and rapidly replaced by CO2 prior to infusing a fecal microbiota transplantation (FMT) material so as not to damage incoming microbiota;
(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;
(xix) a Vitamin C or L-ascorbic acid, optionally administered as a bowel prep, optionally administered before or during a colonic machine or equivalent wash or administered before or during a colonoscopy; optionally administered as an ascorbic acid and sodium ascorbate mixture (optionally administered with a polyethylene glycol or a formulation of: polyethylene glycol 3350, sodium ascorbate, sodium sulfate, ascorbic acid, sodium chloride and potassium chloride as an oral solution,
optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,
and optionally the Vitamin C or L-ascorbic acid is administered orally (optionally wash out luminal fecal material and to dissolve a biofilm simultaneously,
and optionally the Vitamin C or L-ascorbic acid is administered with an enema, optionally at a sodium ascorbate or ascorbic acid formulated or administered in a dose of between about 1 gram (g) to 100 g,
and optionally can be administered as described in (xiv); and/or
(xx) the at least one compound or composition comprises any combination of (i) to (xix).

2. A powder or a lyophilate formulation comprising a dried and powdered formulation, or a lyophilate, of an aqueous liquid formulation of claim 1,
wherein optionally the powder or a lyophilate formulation is capable of being reconstituted as a liquid formulation in an aqueous solution.

3. A product of manufacture for, comprising or containing therein an aqueous liquid formulation of claim 1, wherein optionally the product of manufacture is a container, and optionally the container is a sachet.

4. A method for:
washing or lavaging an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) to remove substantially all or all biofilm that is adherent to the microbiome space (e.g., a gut mucosa or a luminal mucosa), wherein optionally the gut mucosa or luminal mucosa is a colon mucosa,
preparing an individual in need thereof, and optionally the individual is a patient, or a patient's colon, for an in situ microbiome transplantation,
and optionally the in situ microbiome transplantation comprises a fecal microbiota transplantation (FMT), administration, for or infusion or insertion of a living microbe or spore,
wherein optionally the living microbe is a bacterium fungi, Archea organism (Archaebacteria), or bacteriophage, and optionally the microbe or bacterium is a cultured or recombinant microbe or bacterium, or
treating, ameliorating (including decreasing the symptoms of, or decreasing the severity of, or inhibiting progression of) or preventing: a disease, infection or condition caused or exacerbated by an in situ microbiome, and optionally the disease or condition comprises a gastrointestinal (GI) disease, infection or condition or a disease or condition caused by, initiated by or exacerbated by a pathological microbiome, or by a pathological GI or colonic microbiome, or by a pathologic microbial organism,
wherein optionally a pathological microbial organism comprises: a bacteria, bacteriophage, fungi, Archea or virus residing or being housed by a gut biofilm,
and optionally the infection is caused by a Clostridioides bacterium, and optionally the Clostridioides bacterium is C. difficile,
comprising administering or infusing the in situ microbiota transplantation into or onto a tissue or an in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof:
(a) an aqueous formulation or composition as set forth in claim 1,
(b) an aqueous formulation comprising a soap,
wherein optionally the soap comprises a castile soap or equivalent or an IVORY™ soap or equivalent,
and optionally the soap comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide.
and optionally the soap further comprises: sodium tallowate, sodium cocoate, sodium palm kernelate, sodium chloride, sodium silicate, magnesium sulfate or any combination or mixture thereof,
and optionally the soap further comprises: coconut acid, palm kernel acid, tallow acid, palmitic acid, tetrasodium EDTA or any combination or mixture thereof,
and optionally the soap is diluted in water, saline or a super-oxidized aqueous solution, and
optionally the water comprises distilled water, tap water, ozonated water, hydrogenated water, activated or electrolyzed water (optionally comprising sodium hydroxide and/or hypochlorous acid), and optionally the saline comprises a superoxygenated saline or an about 0.9%, or between about 0.5% to 2%, or between about 0.25% to 4%, saline solution,
and optionally the soap comprises a mixture of between about ¼ to 2 ounces (oz), or between about ⅛ to 3 oz, of soap, dissolved or mixed in 1 to 3 quarts, or in about 2 quarts, of water, optionally a distilled water,
(c) an aqueous formulation comprising at least one compound or composition comprising, or selected from the group consisting of:
(i) at least one biofilm disrupting agent comprising at least one enzyme,
wherein optionally the at least one enzyme comprises: a proteinase, a lipase, an amylase, a deoxyribonuclease (DNase), optionally dornase alpha, or PULMOZYME™, an alginase, a lyase or a glycoside hydrolase (optionally dispersin B);
(ii) at least one antibiotic,
and optionally the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,
and optionally the at least one antibiotic is used (or administered) alone (as a single antibiotic) or as a mixture, and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections;
(iii) at least composition selected from the group consisting of: a N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7, nitric oxide, cys-2-decenoic acid, sodium nitroprusside, s-nitroso-l-glutathione (GSNfaO), s-nitroso-N-acetylpenicillamine (SNAP), chlorhexidine, iodine, povidone-iodine (PI) (or WOKADINE™, PYODINE™, BETADINE™), a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or a silver ion, a probiotic (and optionally the probiotic comprises a Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit film formation, and permit antibiotics to be more active), arsenicals, selenium, titanium dioxide, gallium nitrate, an alcohol such as ethanol, hydrogen peroxide, hydrochloric acid, formaldehyde or luminal formalin in low concentrations, ozonated water, hydrogenated water, activated or electrolyzed water, a super-oxidized aqueous solution, nitrofurantoin, hexamine hippurate, potassium hydroxide, mercuric chloride, boric or boronic acid, disodium EDTA, a phytocannabinoid, optionally cannabidiol (CBD), an alkyl dimethylol alkanate (ADMA), or any mixture or combination thereof;
(iv) at least one polyol or a wetting agent,
and optionally the at least one polyol comprises xylitol, sorbitol, mannitol, erythritol, isomalt, maltitol syrup, lactitol, a hydrogenated starch hydrosylate, or mixtures or combinations thereof,
wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof;
(v) at least one surfactant or biosurfactant,
and optionally the at least one biosurfactant comprises: a probiotic, optionally a Bacillus strain, and optionally the Bacillus strain is Bacillus licheniformis,
and optionally the surfactant comprises an anionic, cationic, zwitterionic, or nonionic surfactant, or, any combination thereof,
and optionally the anionic surfactant comprises a sulfate, sulfonate or a phosphate ester,
and optionally the cationic surfactant comprises a tertiary amine or a quaternary ammonium salt,
and optionally the zwitterionic surfactant comprises a phospholipid, and optionally the phospholipid comprises a phosphatidylserine, phosphatidyl-ethanolamine, phosphatidylcholine or a sphingomyelin,
and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,
and optionally the surfactant comprises: a fatty acid esters of a sucrose or a sorbitol; a Tween; an alkyl polyglucoside; an amine or a phosphine oxide; a sulfoxide; or, any combination thereof;
(vi) at least one anti-quorum sensing (QS) compound,
and optionally the at least one QS compound comprises: S-adenosyl-homocysteine, sinefungin, a N-sulfonyl homoserine lactone, or a synthetic derivative thereof, or a mixture or combination thereof; r
(vii) at least one prebiotic,
and optionally the at least one prebiotic comprises: inulin, a chicory extract, a fructan-comprising dietary fiber, N-acetyl glucosamine (NAG), an apple extract such as apple pectin, peas, tomato, rice and garlic or extracts thereof;
(viii) a stain and/or a dye,
wherein optionally the stain or dye comprises Coomassie Brilliant Blue, triarylmethane dye, rhodamine or erythrosine B;
(ix) a stool softening agent or a laxative,
wherein optionally the stool softening agent or laxative comprises: glycerin, sorbitol, lactulose, polyethylene glycol (PEG) (optionally COLYTE™, MIRALAX™), a docusate, a docusate salts or a dioctyl sulfosuccinate (optionally COLACE™, EX-LAX™, SENOKOT S™) or a mixture thereof; or a COLOXYL™ drop;
(x) a charcoal, a carbon or equivalent (e.g., CHARCODOTE™), for example, an activated carbon or charcoal, wherein optionally the carbon or charcoal or equivalent is added at a concentration of between about 1 to 100 grams per liter, and optionally the activated carbon or charcoal is or is formulated as a powdered, granular or extruded activated carbon or charcoal, or is formulated as a bead-activated, woven or polymer-coated carbon;
(xi) an ascorbic acid or fatty acid ester thereof, ascorbyl palmitate, sodium ascorbate, potassium ascorbate, calcium ascorbate or vitamin C, or a liposome comprising the ascorbic acid or fatty acid ester thereof, ascorbyl palmitate, sodium ascorbate, potassium ascorbate, calcium ascorbate or vitamin C, wherein optionally the ascorbic acid or fatty acid ester thereof, ascorbyl palmitate, sodium ascorbate, potassium ascorbate, calcium ascorbate or vitamin C is present in the formulation at a concentration of between about 1 ugm/ml to about 1 gm/ml;
(xii) pure (or substantially pure) or distilled water (H2O) (optionally alkaline water), optionally used alone as a biofilm dissolver exploiting its hypotonic nature to penetrate bacteria resulting in swelling and bursting of the bacteria or other pathogen, and optionally the pure, alkaline or distilled water is used as an enema,
and optionally the enema or a colonic washing is by infusing infused the pure, alkaline or distilled water by use of a colonic machine or equivalent, or by use of a naso-gastric (NG) long tube, or equivalent, and optionally ozone or ozonated water is administered after administration of the pure, alkaline or distilled water;
(xiii) iodine, povidone, povidone-iodine (PI) (or WOKADINE™, PYODINE™ BETADINE™), optionally at a between about 50% to 0.1% concentration delivered to the GI tract; optionally the iodine, povidone, povidone-iodine (PI) is alone or combined with another liquid or a solvent, optionally pure water, and optionally ozone or ozonated water is administered after administration of the pure, alkaline, alkalized or distilled water;
(xiv) an anti-persister cell therapy comprising administration of a compound that can activate a persister cell (optionally a persister bacterial cell) and thereby destroy, neutralize or kill the persister cell by administration (optionally by co-administration) of an antibiotic or a biofilm disrupting or a biofilm-related therapy,
and optionally the anti-persister cell therapy compound and/or the antibiotic or biofilm disrupting or biofilm-related therapy is delivered orally, optionally is ingested as a tablet, geltab or a capsule, optionally for between about 1 to 30 days (d) before a biofilm-disrupting or a biofilm-removal therapy,
and optionally the anti-persister cell therapy compound and/or the antibiotic or biofilm disrupting or biofilm-related therapy is delivered or administered via use of a colonic washout machine or equivalent, or a colonoscope or equivalent, or by use of an overtube or equivalent,
and optionally water, saline, a soap and water mixture, a super-oxidized solution (SOS) (also known as anolyte solution, or oxidative potential water) (optionally as MICRODACYN™ or MICROCYN™), is used as a dissolving or suspending liquid,
and optionally the anti-persister cell therapy compound comprises mitamycin-C, 5-fluorouracil (or ADRUCIL™), cisplatin (or PLATINOL™), cis-2-decenoic acid, dispersin-B (or DspB), a halogenated phenazine (NP) (optionally 2,4-dibrominated HP, or a compound as described in Yang et al Scientific Reports vol 7 (2017) #2003), or equivalents of mixtures thereof,
and optionally the anti-persister cell therapy (to activate resister bacteria in biofilm matrix) comprises pyruvate, a sugar and/or a polyol (optionally a sugar alcohol), and optionally the sugar and/or a polyol comprises mannitol, glucose or fructose or combination thereof;
(xv) a super-oxidized solution (SOS) (also known as anolyte solution, or oxidative potential water) (optionally as MICRODACYN™ or MICROCYN™) optionally used alone as biofilm-removing or biofilm-disrupting agent, optionally administered via a colonic washing machine or equivalent, an overtube or equivalent with colonoscope or equivalent, or via a colonoscope or a nasogastric (NJ) tube or equivalents, and optionally a volume of between about 1 to 36 liters (L) of the solution is used;
(xvi) an ozonated water, optionally used alone as a biofilm-removing or biofilm-disrupting liquid, and optionally is administered using methods described in (xiv) for super-oxidized solutions (SOS);
(xvii) an ozone gas (which may damage a biofilm and/or its resident organisms, and optionally is administered as an insulated gas, optionally administered via a colonoscope or equivalent or by using gas bags or equivalent,
and optionally ozone is administered during a colonoscopy, optionally either in air or with CO2 as insufflating gases, and optionally the ozone gas is substituted or replaced by CO2, and optionally the ozone gas is aspirated and rapidly replaced by CO2 prior to infusing a fecal microbiota transplantation (FMT) material so as not to damage incoming microbiota.
(xviii) N-acetyl-cysteine (NAC), optionally administered alone, optionally administered via or into a rectum (optionally administered as described via methods described in (xiv) above) optionally administered intravenously (IV), optionally administered in high gram doses of between about 250 mg to 50 grams;
(xix) a Vitamin C or L-ascorbic acid, optionally administered as a bowel prep, optionally administered before or during a colonic machine or equivalent wash or administered before or during a colonoscopy;
optionally administered as an ascorbic acid and sodium ascorbate mixture (optionally administered with a polyethylene glycol optionally formulated as a formulation of: polyethylene glycol 3350, sodium ascorbate, sodium sulfate, ascorbic acid, sodium chloride and potassium chloride as an oral solution,
optionally administered at a dose of about 11 gram (g) or more, or optionally administered in a dose of about 45 grams (g), 50 g, or 55 g or more, or at between about 40 g and 60 g, or 30 g and 75 g, or 10 g and 100 g,
and optionally the Vitamin C or L-ascorbic acid is administered orally (optionally wash out luminal fecal material and to dissolve a biofilm simultaneously,
and optionally the Vitamin C or L-ascorbic acid is administered with an enema, optionally at a sodium ascorbate or ascorbic acid formulated or administered in a dose of between about 1 gram (g) to 100 g, and optionally can be administered as described in (xiv); and/or
(xx) the at least one compound or composition comprises any combination of (i) to (xix); or
(d) an aqueous formulation comprising a soap of (b) formulated with or in combination with, or administered with, at least one compound or composition of (c).

5. The method of claim 4, wherein the administering or infusing into the in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises administering or infusing the aqueous formulation of 4(a) or the aqueous formulation or composition of 4(b), 4(c) or 4(d), or a combination thereof, via an oral, nasal or vaginal route or an anal route,
and optionally the aqueous formulation or composition of claim 4 is administered by use of a nasojejunal tube or a rectal speculum or catheter, a colonic tube or an endoscope.

6. The method of claim 4, wherein the administering or infusing into the in situ microbiome space (wherein optionally the in situ microbiome space comprises a gut or colon, sinus, vaginal, oral mucosa, tongue, stomach, skin, bladder, urethral, ureter, ear, bronchial, trachea, pharynx, lung, sinuses, lung or other microbiome space or microbiome-comprising tissue) of an individual in need thereof comprises one, two, three, four, five or six or more applications, washes or lavages of the aqueous formulation of claim 4(a) or the aqueous formulation or composition of claim 4,
and optionally a sufficient amount of the aqueous formulation or composition of claim 4 is administered to remove between about 70% to 100%, 80% to 99.9%, or 85% to 99%, or 90% to 98%, or about 90%, 91%, 92%, 93%, 94%, 95%, 96%, 97%, 98% or 99% or more of the biofilm adherent to the wall or lumen of the in situ microbiome space or a wall or lumen of a gut or a colon,
and optionally between about between about 200 ml to about 1 to 5 liters (L), or about 0.5, 1, 2, 3, 4 or 5 or more liters, of liquid are infused into the individual in need thereof per treatment infusion, or application, wash or lavage,
and optionally each treatment infusion, or application, wash or lavage lasts between about 5 to 10 minutes and 10 hours, for example, between about 1 to 2 hours or 30 to 90 minutes,
and optionally the administering or infusing of a formulation or water into the in situ microbiome space, e.g., gastrointestinal tract, of the individual in need thereof comprises multiple infusions of about 10 cc to about 30 cc every about 1 to 6 hours (hrs), or over a 24 to 48 hours period.

7. The method of any claim 4, wherein the individual in need thereof is a human patient.

8. The method of any claim 4, wherein the administering or infusing into the in situ microbiome space, e.g., gastrointestinal tract, of the individual in need thereof comprises administering the aqueous formulation or composition of claim 4 under pressure or in a liquid pulsating form.

9. The method of claim 4, wherein the administering or infusing into the gastrointestinal tract of an individual in need thereof comprises moving the individual in need thereof or massaging the gut of the individual in need thereof during and/or after administering of the aqueous formulation or composition of claim 4.

10. The method of any claim 4, wherein the administering or infusing into the in situ microbiome space, optionally infusing into a gastrointestinal tract, of the individual in need thereof comprises alternation administering of: (i) water, optionally distilled water or sterile water, and (ii) an aqueous formulation or composition of claim 4,
and optionally the aqueous formulation comprises any one or several of: biofilm disrupting or anti-biofilm agent or reagent; N-acetyl-cysteine (NAC); a super-oxidized solution (SOS); an anti-persister cell therapy compound; iodine or povidone-iodine (PI); a charcoal, a carbon or equivalent; a stool softening agent or a laxative; a Vitamin C or L-ascorbic acid; a prebiotic; an anti-quorum sensing (QS) compound; a polyol or wetting agent; an antibiotic; a stain and/or a dye; ozonated, pure, distilled or alkalized water,
wherein optionally the administering or infusing into the in situ microbiome space, e.g., gastrointestinal tract, of an individual in need thereof comprises one, two, three or more cycles of formulation and water enemas or administrations.

11. A kit comprising an aqueous liquid formulation of claim 1.

12-13. (canceled)

14. A pair of pants or shorts for use with a colonic washout machine, bed or chair, wherein the pair of pants comprises: an orifice or opening sufficient to allow passage of a tube, optionally an enema tube or colonoscopic tube; and a clip, clamp or adhesive (or a plurality of clips, clamps or adhesives) or equivalent thereof capable of securing the tube to the pants or shorts such that the tube does not slip out of or through the orifice or opening, and optionally the pair of pants or shorts is sufficiently tight-fitting such that the pants or shorts can hold the tube in place against a pressure or pulling force on the tube that, but without the tube being secured to the pants or shorts with the fitting, tie, snap, clip, clamp or adhesive or equivalent thereof, the tube would move through the orifice or opening, wherein optionally the pair of pants or shorts is made of, or comprises, an elastic material.

15. A colonic washout machine or device, wherein optionally the colonic washout machine or device is manufactured or configured in the form of a bed or a chair, optionally a movable bed or chair, and the colonic washout machine or device comprises a hole or opening approximate to the position of the anus of an individual sitting or laying on the colonic washout machine or device, wherein the hole or opening is sufficiently large to allow passage of a tube or device, optionally a rectal speculum or catheter, an endoscope, a colonoscope or a colonic tube,
wherein the colonic washout machine or device optionally further comprises:
(a) an auxiliary container or containers, or liquid or formulation holding modules, capable of holding a liquid or formulation attached to the colonic washout machine or device (optionally removably attached) and operatively attached or directly or indirectly connected to an endoscope, colonoscope or rectal speculum or catheter designed for insertion into a colon, and optionally the auxiliary container or containers are operatively attached or connected to the rectal speculum or catheter by use of a second tube,
and optionally the auxiliary container or containers further comprise a value or valves disposed between the auxiliary container or containers and the rectal speculum or catheter, wherein the value or valves are capable of controlling the rate of flow of liquid or formulation from the auxiliary container or containers to the rectal speculum or catheter, or the value or valves are capable of turning on or off the flow of liquid or formulation from the auxiliary container or containers to the rectal speculum or catheter,
and optionally the colonic washout machine or device further comprises or incorporates one or more intake ports, cassette spaces or cavities, each which can take (or input) a cassette, cartridge or a removable cartridge housing, and optionally the removable cartridge housing is fitted to have inserted therein a cassette or a cartridge,
and optionally the cassette or cartridge comprises or has contained therein a liquid, formulation, powder or lyophilate formulation for dissolving a biofilm; a drug or an active agent; or, an FMT material,
and optionally the colonic washout machine or device further comprises a pump or pumps operably connected to the rectal speculum or catheter, the auxiliary container or containers, or the second tube to move or facilitate the flow of liquid or formulation contents of the auxiliary container or containers out to a liquid or formulation flowing through the rectal speculum or catheter,
and optionally the pump or pumps are capable or programmed to create a water hammer effect, which is created by a rapid back and forth movement of a water or liquid or formulation column for a short distance, for example, for a distance of between about 0.5 to 10 cm, or between about 1 mm to 5 cm, and the pump or pumps comprise or use a piston-driven system or equivalent such that a water or liquid column is induced to have a back-and-forth movement in situ,
and optionally the colonic washout machine or device further comprises a warming module, heater or heating unit to heat a fluid or liquid or formulation in the auxiliary container or containers, or the tubes, to about body temperature,
and optionally the device (optionally the one or more intake ports, or cavities, or cassette or cartridge housings) further comprise an thermometer or thermostat capable of reading the temperature of a liquid or formulation, and thermometer or thermostat are capable of controlling the temperature of the liquid or formulation, where the liquid or formulation or fluid can be warmed and/or maintained at approximate body temperature, or the liquid or formulation or fluid can be warmed and/or maintained at about 37° C.,
and optionally the thermometer or thermostat is remotely read and/or is remotely controlled, for example, by a hand-held device or a computer,
and optionally the auxiliary container or containers, the cassette or cartridge, the cassette or cartridge housing, or the second tube, comprise a filter or filters capable of separating or straining a particulate matter from a liquid or formulation before it is infused into the colon, and optionally the auxiliary container or containers, the cassette or cartridge, the cassette or cartridge housing, or the second tube, comprise fitting that allow removal or exchange of or insertion of a new filter or filters;
(b) a master container or containers capable of holding liquids or formulations for infusion into a colon, wherein optionally the master container or containers are operatively connected to the auxiliary container or containers of (a), and the master container or containers are operatively attached or connected to a rectal speculum or catheter, and optionally the master container or containers are operatively attached or connected to the rectal speculum or catheter by use of a third tube,
and optionally the master container or containers further comprise a value or valves disposed between the master container or containers and the rectal speculum or catheter, wherein the value or valves are capable of controlling the rate of flow of liquid or formulation from the master container or containers to the rectal speculum or catheter, or the value or valves are capable of turning on or off the flow of liquid or formulation from the master container or containers to the rectal speculum or catheter,
and optionally the colonic washout machine or device further comprises a pump operably connected to the rectal speculum or catheter, the master container or containers, or the third tube to move or facilitate the flow of liquid or formulation contents of the master container or containers out to a liquid or formulation flowing through the rectal speculum or catheter,
and optionally the colonic washout machine or device further comprises a heater or heating unit to heat the liquid or formulation in the master container or containers to about body temperature, or to about 37° C.,
and optionally the master container or containers or the third tube comprise a filter or filters capable of separating or straining particulate matter from a liquid or formulation before it is infused into the colon;
(c) a pump operably connected to the rectal speculum or catheter, the master container or containers, the third tube, the auxiliary container or containers and/or the second tube, where the pump when operational (turned on) moves or facilitates the flow of liquid or formulation contents of the rectal speculum or catheter, the master container or containers, the third tube, the auxiliary container or containers and/or the second tube, to the colon,
and optionally the pump is a low pressure pump and/or a pressure adjustable pump, and optionally the pump further comprises a pressure gauge and pressure readings from the pump pressure gauge are transmitted to or are displayed to a reading device or screen on the colonic washout machine or device or remotely to a computer or a portable device, and optionally the portable device is a hand-held device or a smart phone, and optionally the pump is controlled remotely by use of a computer or a portable device or a foot pedal,
and optionally the pump is capable of providing a pulsating movement of liquid or formulation through the rectal speculum or catheter into the colon, and optionally the pulsating movement of the pump is controlled remotely by use of a computer or a portable device or a foot pedal,
(d) a first set of motor or motors operably connected to rollers or equivalent for massaging, shaking or vibrating an individual lying on or sitting in the colonic washout machine or device,
and optionally the first set of motor or motors are controlled remotely by use of a computer or a portable device or a foot pedal,
(f) a second set of motor or motors operably connected to the colonic washout machine or device and capable of moving the colonic washout machine or device in a tipping or side to side movement,
and optionally the second set of motor or motors are controlled remotely by use of a computer or a portable device or a foot pedal; or
(g) any combination of (a) to (f).

16. A rectal infusion or rectal aspiration tube or speculum comprising a first or distal end for inserting into the colon of an individual and a second proximal end comprising a fitting for connection to a container or source of liquid or formulation for infusion into the colon, wherein the first end of the rectal infusion or rectal aspiration tube comprises a plurality of exit holes, orifices or openings to allow passages of fluids,
wherein the plurality of exit holes, orifices or openings are between about, or average from between about, 2 mm to 20 cm, and optionally the plurality of exit holes, orifices or openings are milled or made by a 3D printer,
and optionally the plurality of openings are positioned at about 1, 2, and 3 cm from the distal tip, or a plurality of openings are positioned between about 0.5 to 20, or 1 to 10 cm from the distal tip of the rectal infusion or rectal aspiration tube, or speculum.

17. The aqueous liquid formulation of claim 1, wherein:
the soap is between about 1% to 95%, 5% to 90%, 10% to 80%, 15% to 70%, 20% to 60%, or 30% to 50%, by weight or volume of the total aqueous liquid formulation,
the soap comprises a castile soap or equivalent or an IVORY™ soap or equivalent,
the soap comprises: (i) an oil, optionally one or more vegetable or plant-extracted oils, and optionally the vegetable or plant-extracted oil comprises: coconut oil, olive oil, hemp oil, jojoba oil, laurel oil, or a mixture or combination thereof; and, (ii) an alkali, wherein optionally the alkali comprises potassium hydroxide or sodium hydroxide,
the soap further comprises: sodium tallowate, sodium cocoate, sodium palm kernelate, sodium chloride, sodium silicate, magnesium sulfate or any combination or mixture thereof,
the soap further comprises: coconut acid, palm kernel acid, tallow acid, palmitic acid, tetrasodium EDTA or any combination or mixture thereof, the soap is diluted in water, saline or a super-oxidized aqueous solution (optionally OXUM™, MICRODACYN™, DERMACYN™),
the water comprises distilled water, tap water, ozonated water, a hydrogen water (wherein optionally the hydrogen water is made by infusing hydrogen gas into water under pressure, and optionally the hydrogen water has between about 5 mg to 10 mg hydrogen per liter of water), an activated or electrolyzed water (optionally comprising sodium hydroxide and/or hypochlorous acid),
the saline comprises a superoxygenated saline or an about 0.9%, or between about 0.5% to 2%, or between about 0.25% to 4%, saline solution, and/or
the soap comprises a mixture of between about ¼ to 2 ounces (oz), or between about ⅛ to 3 oz, of soap, dissolved or mixed in 1 to 3 quarts, or in about 2 quarts, of water, optionally a distilled water.

18. The aqueous liquid formulation of claim 1, wherein the at least one enzyme in the at least one biofilm disrupting agent comprises: a proteinase, a lipase, an amylase, a deoxyribonuclease (DNase), optionally dornase alpha, an alginase, a lyase or a glycoside hydrolase (optionally dispersin B).

19. The aqueous liquid formulation of claim 1, wherein the at least one antibiotic comprises: a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof, or optionally the combination secnidazole, nitazoxanide and furazolidone,
and optionally the at least one antibiotic is used (or administered) alone (as a single antibiotic) or as a mixture,
and optionally the antibiotic is administered orally or via a nasogastric (NG) tube or via an enema, and optionally the at least one antibiotic is administered prior to commencing colonic biofilm removal (wherein the colonic biofilm removal is done by purging), to minimize or substantially diminish the presence of one or more intra-biofilm infections.

20. The aqueous liquid formulation of claim 1, wherein the at least one polyol or a wetting agent comprises xylitol, sorbitol, mannitol, erythritol, isomalt, maltitol syrup, lactitol, a hydrogenated starch hydrosylate, or mixtures or combinations thereof,
wherein optionally the wetting agent comprises a polyethylene (PEG), bisoxatin (optionally comprising 10 mg to 3 grams bisoxatin), bisacodyl (optionally comprising 0.5 mg to 50 mg bisacodyl) or mixtures thereof.

21. The aqueous liquid formulation of claim 1, wherein the at least one surfactant or biosurfactant comprises: a probiotic, optionally a Bacillus strain, and optionally the Bacillus strain is Bacillus licheniformis,
and optionally the surfactant comprises an anionic, cationic, zwitterionic, or nonionic surfactant, or, any combination thereof,
and optionally the anionic surfactant comprises a sulfate, sulfonate or a phosphate ester,
and optionally the cationic surfactant comprises a tertiary amine or a quaternary ammonium salt,
and optionally the zwitterionic surfactant comprises a phospholipid,
and optionally the phospholipid comprises a phosphatidylserine, phosphatidyl-ethanolamine, phosphatidylcholine or a sphingomyelin,
and optionally the nonionic surfactant comprises; a fatty acid ester of a polyhydroxy compound or glycerol; a poloxamer; an ethoxylate (optionally a fatty acid ethoxylate); or, a polyethoxylated amine, monoethanolamine or diethanolamine,
and optionally the surfactant comprises: a fatty acid esters of a sucrose or a sorbitol; a Tween; an alkyl polyglucoside; an amine or a phosphine oxide; a sulfoxide; or, any combination thereof.

22. The aqueous liquid formulation of claim 1, wherein the at least one QS compound comprises: S-adenosyl-homocysteine, sinefungin, a N-sulfonyl homoserine lactone, or a synthetic derivative thereof, or a mixture or combination thereof.
